(19) United States
(12) Patent Application Publication
(10) Pub. No.: US 2026/0069921 A1
(43) Pub. Date: Mar. 12, 2026

(54) INFORMATION PROCESSING APPARATUS, INFORMATION PROCESSING SYSTEM, PROGRAM, AND METHOD

(71) Applicant: Tsutomu KAWASE, Kanagawa (JP)

(72) Inventor: Tsutomu KAWASE, Kanagawa (JP)

(73) Assignee: Ricoh Company, Ltd., Tokyo (JP)

(21) Appl. No.: 19/306,364

(22) Filed: Aug. 21, 2025

(30) Foreign Application Priority Data
Sep. 11, 2024 (JP) 2024-157146

(51) Int. Cl. A63B 24/00; A63B 71/06; G06F 40/40; G06T 11/60; G11B 27/00; G11B 27/036

(52) U.S. Cl. CPC A63B 24/0062; A63B 71/0622; G06F 40/40; G06T 11/60; G11B 27/005; G11B 27/036; A63B 2220/05; A63B 2220/803; A63B 2220/808; A63B 2220/836; A63B 2225/50

(57) ABSTRACT

An information processing apparatus includes: circuitry configured to input motion information related to a physical exercise of a user; input reaction information related to a reaction of the user; and output utterance information related to exercise support for the user, the utterance information being generated based on the reaction information and the motion information.

### CROSS-REFERENCE TO RELATED APPLICATIONS

The present application is based on and claims priority to Japanese Patent Application No. 2024-157146 filed on Sep. 11, 2024, the entire contents of which are hereby incorporated by reference.

### BACKGROUND

### 1. Field of the Invention

The present disclosure relates to information processing, and more particularly to an information processing apparatus, an information processing system, a program, and a method.

### 2. Description of the Related Art

An utterance promotion device which can be used for cognitive function training even in a small group is known. In order to promote light exercise and brain activity of users such as the elderly, an exercise support system which provides field experience by video and sound is known.

### SUMMARY

The present disclosure provides an information processing apparatus having the following features in order to solve the above issues. The information processing apparatus includes a motion information inputter to which motion information related to a physical exercise of a user is input. The information processing apparatus also includes a reaction information inputter to which reaction information related to a reaction of the user is input. The information processing apparatus further includes an utterance information outputter configured to output utterance information related to the exercise support for the user based on the reaction information that is input to the reaction information inputter and the motion information that is input to the motion information inputter.

### BRIEF DESCRIPTION OF THE DRAWINGS

FIG. 1 is a schematic diagram illustrating an exercise support system according to one or multiple embodiments of the present disclosure;

FIG. 2 is a diagram illustrating functional blocks of an exercise support system according to one or multiple embodiments of the present disclosure;

FIG. 3A is a diagram illustrating an exemplary screen displayed on a display of the exercise support system according to one or multiple embodiments of the present disclosure;

FIG. 3B is a diagram illustrating an exemplary screen displayed on the display of the exercise support system according to one or multiple embodiments of the present disclosure;

FIG. 3C is a diagram illustrating an exemplary screen displayed on the display of the exercise support system according to one or multiple embodiments of the present disclosure;

FIG. 3D is a diagram illustrating an exemplary screen displayed on the display of the exercise support system according to one or multiple embodiments of the present disclosure;

FIG. 4A is a diagram illustrating an exemplary data structure of speech information generated by the exercise support system according to one or multiple embodiments of the present disclosure;

FIG. 4B is a diagram illustrating an exemplary data structure of motion information generated by the exercise support system according to one or multiple embodiments of the present disclosure;

FIG. 4C is a diagram illustrating an exemplary data structure of biometric information generated by the exercise support system according to one or multiple embodiments of the present disclosure;

FIG. 4D is a diagram illustrating an exemplary data structure of camera information generated by the exercise support system according to one or multiple embodiments of the present disclosure;

FIG. 4E is a diagram illustrating an exemplary data structure of image attribute information generated by the exercise support system according to one or multiple embodiments of the present disclosure;

FIG. 4F is a diagram illustrating an exemplary data structure of user information generated by the exercise support system according to one or multiple embodiments of the present disclosure;

FIG. 5 is a schematic diagram illustrating a learning process of a machine learning model in the exercise support system according to one or multiple embodiments of the present disclosure;

FIG. 6 is a flowchart illustrating walking exercise support processing executed by the information processing apparatus in the exercise support system according to one or multiple embodiments of the present disclosure;

FIG. 7 is a diagram illustrating utterance information generation processing based on motion information and speech information, executed by the information processing apparatus according to a first embodiment of the present disclosure;

FIG. 8 is a table illustrating conversation examples generated based on the motion information and the speech information, executed by the information processing apparatus according to the first embodiment of the present disclosure;

FIG. 9 is a diagram illustrating the utterance information generation processing based on the motion information, the speech information, and the image attribute information, executed by the information processing apparatus according to a second embodiment of the present disclosure;

FIG. 10 is a table illustrating conversation examples generated based on the motion information, the speech information, and the image attribute information, executed by the information processing apparatus according to the second embodiment of the present disclosure;

FIG. 11 is a diagram illustrating video playback control based on the motion information, executed by the information processing apparatus according to a third embodiment of the present disclosure;

FIG. 12 is a diagram illustrating abnormality report processing based on biometric information, executed by the information processing apparatus according to a fourth embodiment of the present disclosure;

FIG. 13 is a diagram illustrating the video content proposal processing executed by the information processing apparatus according to a fifth embodiment of the present disclosure;

FIG. 14 is a diagram illustrating a user screen displayed on the display of the exercise support system according to another embodiment of the present disclosure;

FIG. 15 is a diagram illustrating functional blocks of an example of distributed implementation of the exercise support system according to an embodiment of the present disclosure;

FIG. 16 is a schematic diagram illustrating an exercise support system 100a according to a first modified example;

FIG. 17 is a diagram illustrating a configurational example of an exercise support system 100b according to a second modified example;

FIG. 18 is a block diagram illustrating an example of a hardware configuration of an information processing apparatus;

FIG. 19 is a schematic diagram illustrating the exercise support system according to yet another embodiment of the present disclosure;

FIG. 20 is a diagram illustrating functional blocks of the exercise support system according to the yet another embodiment of the present disclosure; and

FIG. 21 is a schematic diagram illustrating the exercise support system according to still another embodiment of the present disclosure.

### DETAILED DESCRIPTION OF THE PRESENT DISCLOSURE

The present disclosure has been made in consideration of the above issues, and it is an object of the present disclosure to support exercise of a user by providing a suitable conversation by taking into account an exercise condition of the user.

An information processing apparatus, an information processing system, a method, and a program according to an embodiment of the present disclosure will be described in detail in the following with reference to the drawings. However, the information processing apparatus, the information processing system, the method, and the program according to the embodiment of the present disclosure are not limited to those described in the following. In the following description, the same or similar members or functions are referred to by the same names and symbols, and the detailed description is omitted as appropriate.

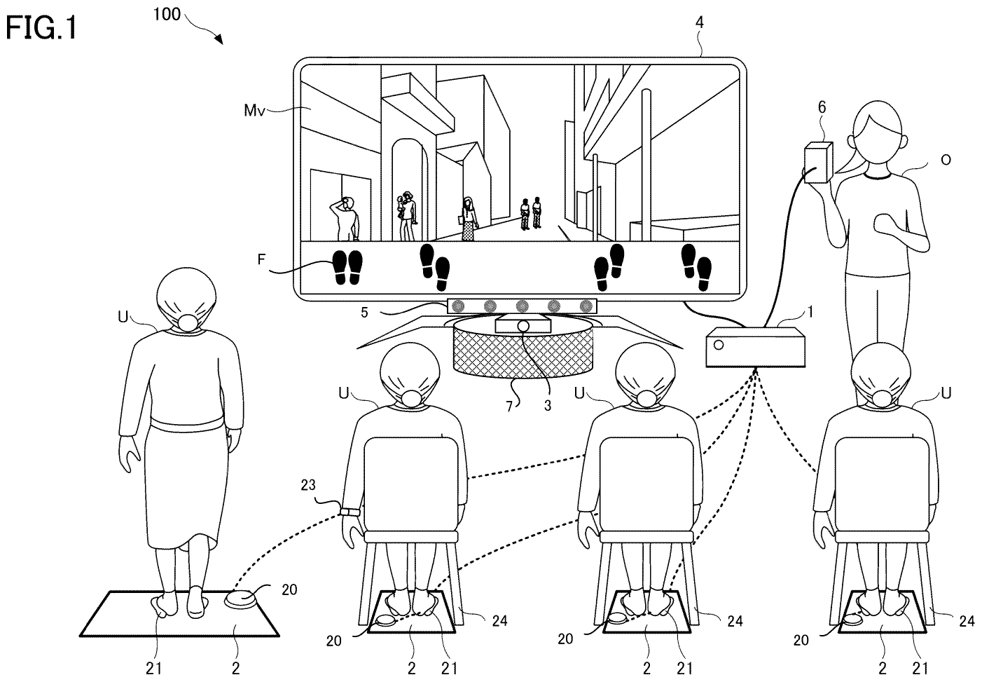

With reference to FIGS. 1 to 6, the overall configuration of an exercise support system 100 as the information processing apparatus or information processing system according to one or multiple embodiments of the present disclosure will be described in the following.

FIG. 1 is a schematic diagram illustrating the exercise support system 100 according to one or multiple embodiments of the present disclosure. The exercise support system 100 is a system for promoting a physical exercise of a user U. In the embodiment to be described, a case in which “walking exercise” is performed as the physical exercise will be described as an example, where the “walking exercise” includes a stepping exercise in which the foot is raised and lowered in a predetermined position. The walking exercise is a preferred example of the physical exercise to which the control according to the embodiment of the present disclosure can be suitably applied, but it is not necessarily limited thereto.

As illustrated in FIG. 1, the exercise support system 100 includes an information processing apparatus 1 and a gait sensor 20 (an example of a motion information obtaining device) configured to output motion information related to the walking exercise of the user U. The environment of the user U may further include a mat 2 on which the user U performs the walking exercise. The mat 2 may include a printed mark 21 which serves as an indication of the position to step on. The gait sensor 20 is disposed on or adjacent to the mat 2, and is configured to detect a state of the left and right feet of the user U on the mat 2, as well as to output motion information (indicating landing, leaving, stepping, stopping, etc.) related to the landing or leaving of the feet of the user U relative to the mat 2 or the walking or stopping of the user U to the information processing apparatus 1 as the motion information.

In the embodiment to be described, it is assumed that the gait sensor 20 separated from the mat 2 is used. However, the configuration of the gait sensor 20 is not particularly limited, and may be integrated with the mat 2, for example, a stepping detection mat using a pressure-sensitive sheet may be used. As for the configuration of the gait sensor 20 configured to detect the walking exercise, various configurations can be used, and thus no further explanation will be provided.

The exercise support system 100 as illustrated in FIG. 1 is configured such that it can be used in parallel by a plurality of users U. In the example as illustrated in FIG. 1, four users U are using the exercise support system 100, and four sets of the mat 2 and the gait sensor 20 are prepared so as to correspond to the four users U. The information processing apparatus 1 is configured to obtain the motion information of each of the four users U from each of the four gait sensors 20.

In the example as illustrated in FIG. 1, one user U is performing the walking exercise while standing, and the other three users U are performing the walking exercise while sitting in their chairs 24. Thus, in the exercise support system 100 according to the present disclosure, the user U can choose whether to perform the walking exercise while standing or sitting according to the preference and health condition of the user U.

The information processing apparatus 1 is configured to obtain information on a walking state and walking pace of the user U from the motion information that is input from the gait sensor 20. The information processing apparatus 1 can calculate a walking pace of the user U from a landing interval at which the foot of the user U touches the mat 2 or the like based on the motion information. Alternatively, the walking pace (the length of time between stepping on the mat 2 with one foot and stepping on the mat 2 with the other foot) can be calculated instead of the landing interval at which the foot touches the mat 2.

The exercise support system 100 as illustrated in FIG. 1 further includes a display 4 on which a video Mv is displayed. The display 4 is a display device such as a liquid crystal display, an organic electroluminescence (EL) display, and a plasma display. The information processing apparatus 1 performs control to change the playback speed of the video Mv displayed on the display 4 according to the walking pace of the user U. More specifically, the information processing apparatus 1 can control the playback of the video Mv displayed on the display 4 according to the exercise condition of one or more users U detected by one or more gait sensors 20. In the exercise support system 100 as illustrated in FIG. 1, by displaying the video Mv viewed by each of the four users U on a single display 4, the video viewed by each user U can be shared among the four users U.

In the embodiment to be described, it is assumed that a plurality of users U perform the walking exercise within a detection range of the gait sensor 20 on the mat 2 at each position while viewing the single display 4. The exercise support system 100 can control the playback of the video Mv while the user U performs the walking exercise, and the playback of the video Mv can be stopped when the user U stops the walking exercise. However, when the exercise support system 100 is used by the plurality of users U, it is difficult to play back the video according to the speed of each user U when there is only one display 4. In addition, there is a case where the user U stops the walking exercise due to fatigue.

Therefore, the playback of the video may be controlled according to the walking state of a majority of the users U. For example, the playback may be advanced when the majority of the users U are engaged in the walking exercise (or when even one of the users U is engaged in the walking exercise), and the playback may be stopped when the majority of the users stop walking (or all of them stop walking). Then, the playback may be resumed when the majority of the users U start the walking exercise (or when any one of the users U starts the walking exercise). In addition, foot marks F, which display stepping states of the users U, are displayed on the display 4 and are changed in accordance with the stepping of each user U, such that the stepping states of the plurality of users U can be confirmed with each other, and the users U can talk to each other or encourage each other.

The user U can visually check the video Mv, which is played back according to the walking state of the user U, through the display 4, such that the user U can have a simulated experience as if the user U is walking in a sightseeing area (for example, by watching video content shot while walking around the sightseeing area). Especially, in the embodiment as illustrated in FIG. 1, since the plurality of users U can use the display 4 in parallel, the simulated experience such as walking can be shared among the users U. Therefore, the user U can work on exercise while having more fun than when using the exercise support system by the user alone. When the exercise support system 100 is used in parallel by the plurality of users U, the users U can perform an exercise that leads to rehabilitation while recalling and talking about the scene and sharing their impressions among the plurality of users U.

It should be noted that in the described embodiment, the configuration is such that the plurality of users U can use the display 4 in parallel, but the configuration of the exercise support system 100 is not limited and may be constructed as a system for a single user.

In the example as illustrated in FIG. 1, a part or all of the users U may be provided with a biosensor 23 (an example of a reaction information obtaining device) such as a smart device having functions such as a heart rate monitor, a blood oxygen level measurer, and an activity level meter, etc., a smartwatch, a pedometer (registered trademark), etc., in addition to the gait sensor 20. The biosensor 23 measures a heart rate, a blood oxygen level, an activity level, etc. of a wearer, and outputs them to the information processing apparatus 1 as biometric information. The information processing apparatus 1 may receive the biometric information from the biosensor 23, and perform control according to the biometric information when playing back a video.

The exercise support system 100 illustrated in FIG. 1 further includes a camera 3 configured to take a picture including the use environment and the one or more users U by including them in the field of view of the camera 3. The camera 3 may be provided separately from the information processing apparatus 1 as illustrated in FIG. 1, or may be provided integrally with the information processing apparatus 1. The camera 3 can be used to identify a user. The camera 3 detects a face area from a photographed image and outputs a face image or an image feature of the user U to the information processing apparatus 1. The information processing apparatus 1 identifies the user U from the face image or the image feature, and can respond to the user U individually when playing back a video. The camera 3 also detects a direction of the gaze of the user U by specifying the position of an image region of the iris included in the face image of the user U photographed by the camera 3 by an image processing circuit, and the information processing apparatus 1 may perform control corresponding to the direction of the gaze when playing back the video.

The exercise support system 100 illustrated in FIG. 1 further includes a speaker 5 configured to generate sound. The speaker 5 may be provided separately from the information processing apparatus 1 as illustrated in FIG. 1, or may be provided integrally with the information processing apparatus 1. The exercise support system 100 is configured to generate sound from the speaker 5 in accordance with the video Mv displayed on the display 4. For example, when the video Mv includes a scene that is viewed when walking on a cobblestone pavement, the exercise support system 100 generates the sound of footsteps walking on the cobblestone pavement from the speaker 5. Thus, the user U can have a more realistic simulated experience by using the auditory sense. The speaker 5 can also output a predefined narration or the like. The speaker 5 can also output as speech the utterance information generated by the information processing apparatus 1 by performing the processing described in the following in detail. The speaker 5 has, for example, a multi-channel (for example, 5.1 channel) sound field creation function, and can generate sound with directivity.

In the example as illustrated in FIG. 1, the exercise support system 100 further includes a microphone array 7 (an example of the reaction information obtaining device) which is a sound collector for collecting sound. The microphone array 7 may be provided separately from the information processing apparatus 1 as illustrated in FIG. 1, or may be provided integrally with the information processing apparatus 1. The exercise support system 100 receives speech input from the surrounding environment via the microphone array 7. By applying a beamforming technology, the microphone array 7 is configured to decompose the sound into sounds by incoming directions and obtain speech signals by directions. Thus, it is possible to recognize the utterance of the user U even in a noisy and busy environment. Since the microphone array 7 obtains the speech signals by directions, it is also possible to distinguish the user who speaks for each direction (for example, assuming that utterances from the same direction belong to the same user). By using the microphone array 7, it is expected that the exercise support system 100 can distinguish the words uttered simultaneously by multiple people.

The user U can receive motion stimuli, visual stimuli, auditory stimuli, etc., through the video Mv which changes according to the motion information from the gait sensor 20, and can perform a walking exercise while enjoying the scenery. The stimulation to be given to the user U is not limited to those of the above-described embodiment. In other embodiments, other sensory stimuli such as tactile stimuli and olfactory stimuli may be provided in addition to the above-described images and sounds. For example, in addition to the display 4 and the speaker 5, other external devices such as a lighting device, an air conditioner, an odor generator, and a blower may be provided. The lighting device, the air conditioner, the odor generator, and the blower perform actions that act on the senses of the user U. The sense is a function or consciousness of feeling an external stimulus, for example, at least one of a person's sense of sight, hearing, smell, or touch. The actions that act on the senses of the user may include actions related to at least one of illuminance, wind, smell, water droplets, smoke, or air temperature around the user U. The illuminance can be controlled by the lighting device, the wind can be controlled by the blower, the smell can be controlled by the odor generator, and the air temperature can be controlled by the air conditioner. A part of the speech emitted from the speaker 5, the control of other external devices such as the lighting device, the air conditioner, the odor generator, and the blower can be defined in a script prepared in advance with the video data.

In FIG. 1, an operator O is illustrated separately from the user U. The operator O is, for example, a caregiver who takes care of a person to be cared for as the user U. The operator O holds a remote controller 6, and the exercise support system 100 is operable via the remote controller 6. By operating the exercise support system 100 by the operator O by using the remote controller 6, the labor of operation by the user U can be reduced and the operation of the exercise support system 100 can be smoothly performed. Thus, the user U is motivated to use the exercise support system 100, and the exercise of the user U can be promoted.

The operator O uses the remote controller 6 to select the video content to be displayed on the display 4 during the walking exercise, and to give instructions such as start, stop, or restart of the video contents. The remote controller 6 can be a touch panel, an operation button, a keyboard, a joystick, or a combination thereof. The remote controller 6 may be a remote controller detachable from the exercise support system 100 or the information processing apparatus 1. Alternatively, the remote controller 6 may be provided integrally with the information processing apparatus 1. The remote controller 6 may be an information processing terminal such as a tablet or a smartphone.

Hereinafter, the exercise support system 100 described above will be described in more detail assuming that it is used in a facility for the elderly.

In the exercise support by using the exercise support system 100, the operator O, such as a caregiver or care staff member, performs basic operations such as selecting the video contents and instructing playback, to start the walking exercise (for example, recreation and rehabilitation). However, it may be difficult for the user to maintain interest in performing the walking exercise while simply watching the video. Therefore, it is desirable for the operator O to talk to the user, expand conversation, or encourage the user to continue the exercise during the walking exercise. In contrast to this, the caregiver or care staff member who has other duties does not have much time to devote to the work other than the primary care work. Therefore, it is expected to reduce the burden on the supporter by using a dialogue generation system in the exercise support system 100. However, when communicating between the dialogue generation system and the user, it is difficult to provide sufficient exercise support to the user, because generation of a dialogue based only on the user's utterance cannot generate a suitable dialogue corresponding to the exercise condition of the user.

Therefore, the exercise support system 100 according to the present embodiment aims to achieve the exercise support for the user by automatically generating utterance information according to a state of engagement of the user U on the walking exercise and providing a dialogue suitable for the exercise condition. Here, the exercise condition means the condition (state) of the user who performs physical exercise, such as the amount of exercise related to the physical exercise of the user and the reaction of the user accompanying the physical exercise.

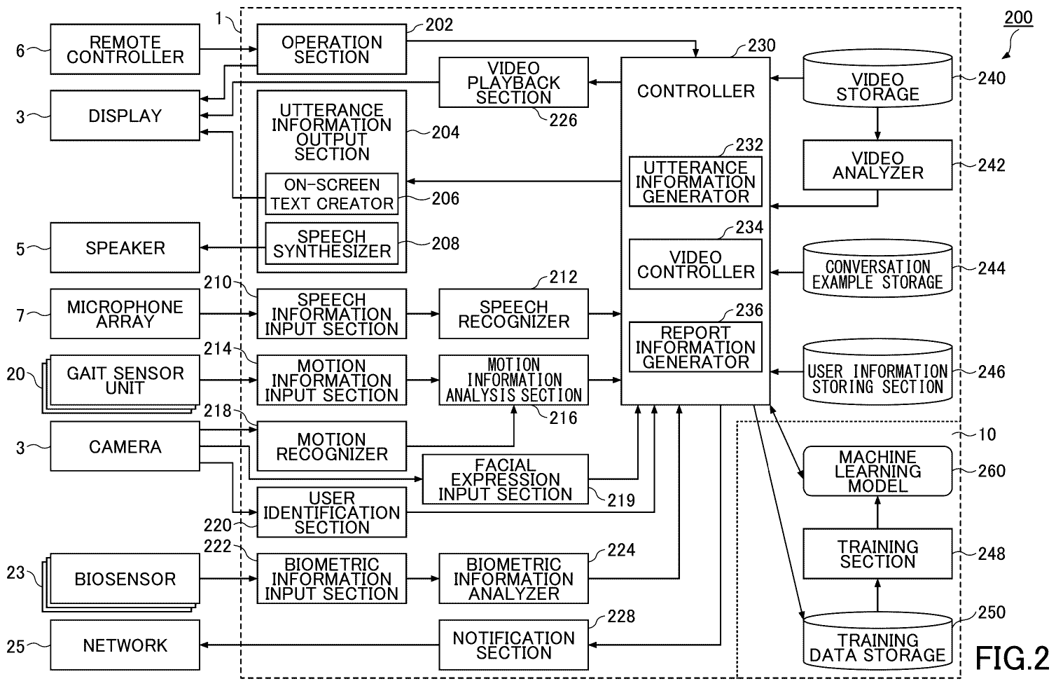

The functional configuration of the exercise support system 100 for achieving the above purpose will be described more specifically in the following with reference to FIG. 2. FIG. 2 is a diagram illustrating a functional block 200 of the exercise support system 100 according to one or multiple embodiments of the present disclosure.

The functional block 200 illustrated in FIG. 2 includes an operation section 202, an utterance information output section 204, a speech information input section 210, a speech recognizer 212, a motion information input section 214, a motion information analysis section 216, a motion recognizer 218, a facial expression input section 219, a user identification section 220, a biometric information input section 222, a biometric information analyzer 224, a video playback section 226, a notification section 228, a controller 230, a video storage 240, a video analyzer 242, a conversation example storage 244, and a user information storing section 246.

The controller 230 performs overall processing and control for supporting the exercise of the user U, including processing for generating utterance information to be described in the following. The controller 230 generates utterance information based on input information that is input via input sections such as the speech information input section 210, the motion information input section 214, the facial expression input section 219, and the biometric information input section 222, and outputs the utterance information to the utterance information output section 204. The controller 230 can also control the video playback by the video playback section 226 based on the input information. The controller 230 can also store the contents of conversation performed during the walking exercise, the state of engagement in the walking exercise, and topic information extracted from the conversation contents, in the user information storing section 246, which will be described in the following, according to the state of engagement in the walking exercise by the user U.

The operation section 202 receives input from the remote controller 6 (operation device) operated by the operator O, and receives selection of the video contents to be played back during the walking exercise. The operator O inputs, through the remote controller 6, other information relating to the registration of the participating user U and the gait sensor 20, such as which gait sensor 20 is used by each participating user U, and the operation section 202 transmits the information to the controller 230. The registration of the participating user U may be performed in part or in whole by the user identification by using the camera 3 described above. Similarly, the correspondence between the user U and the gait sensor 20 may be performed in part or in whole by designating the user by speech and having the user perform the stepping exercise.

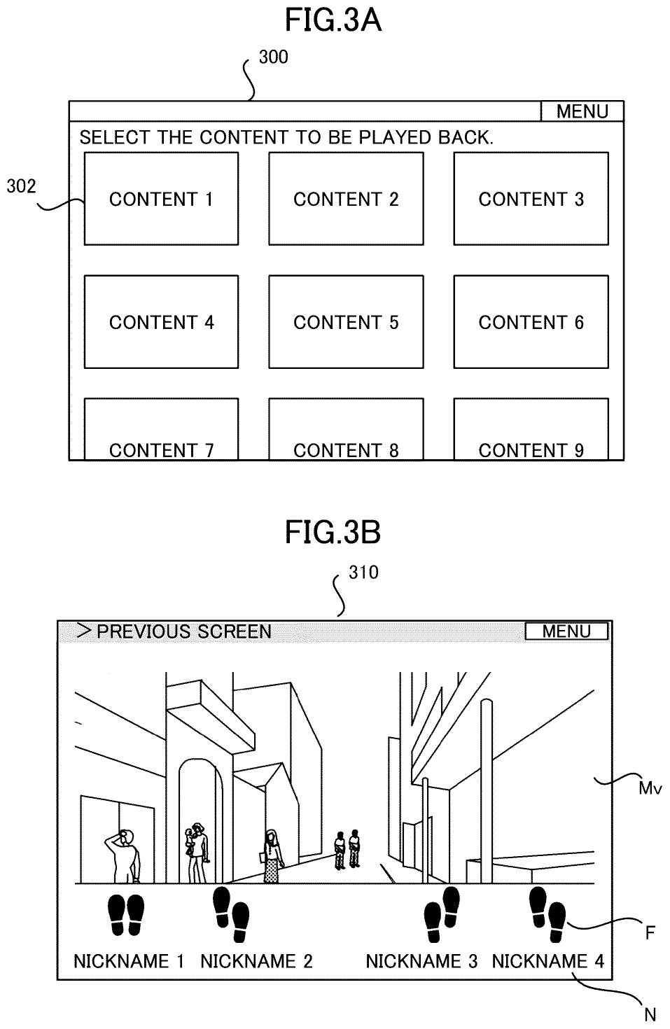

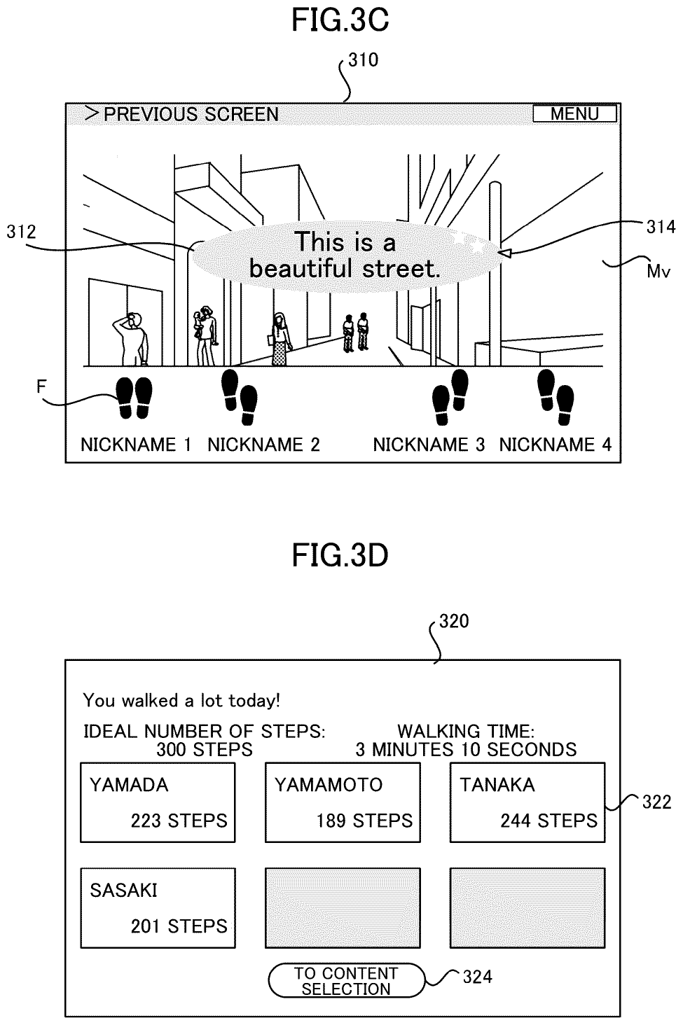

FIGS. 3A to 3D illustrate exemplary screens displayed on the display 4 of the exercise support system 100 according to one or multiple embodiments of the present disclosure. In FIG. 3A, a content selection screen 300 for selecting video contents to be played back from among a plurality of video contents prepared for supporting the walking exercise is illustrated. The operation section 202 outputs an operation screen for the operator o to operate by using the remote controller 6, and determines an instruction from the operator O based on the operation performed by the remote controller 6 and the contents of the output operation screen. In the content selection screen 300 as illustrated in FIG. 3A, thumbnails 302 of a plurality of video contents are arranged in a tile-like arrangement, and the operator O can operate the remote controller 6 and select the video contents to be played back via the operation section 202. The plurality of prepared video contents are stored in the video storage 240. In the content selection screen 300, the selection of video contents may be rearranged or narrowed to select a specific video content according to, for example, the past history information (contents of conversation and the state of engagement in the walking exercise, in the past walking exercise) of the participating user U stored in the user information storing section 246 described in the following. In addition, instead of the operation by the remote controller 6, the video contents to be played back may be selected by speech recognition (speech recognition of the content name, cursor movement by speech recognition, etc.).

When the video contents to be played back are selected, as illustrated in FIG. 3B, a user screen 310 schematically showing the walking state of each user with the foot mark F while playing back the video Mv is displayed. The controller 230 reads the video contents selected via the operation section 202 from the video storage 240 and causes the video playback section 226 to play back the video. The video playback section 226 is included in the image information outputter in the present embodiment. Here, it is assumed that the user starts the walking exercise when the user screen 310 is displayed. In the user screen 310, the name N of the identified user, which will be described in the following, and an animation of the foot mark F that is pseudo walking is displayed in response to the exercise condition of each user.

Referring again to FIG. 2, the utterance information output section 204 receives the utterance information (text) generated by the controller 230, and executes output processing to output the utterance information to the output sections such as the display 4 and the speaker 5. More specifically, the utterance information output section 204 includes an on-screen text creator 206 and a speech synthesizer 208.

The display 4 can display an on-screen text or a subtitle corresponding to the utterance information superimposed on the video Mv to be played back or in a subtitle portion in a lower part of the video. The on-screen text creator 206 creates an on-screen text in which a predetermined font is set based on the utterance information from the controller 230, adds images such as illustrations as necessary, and displays them on the screen of the display 4.

In FIG. 3C, the user screen 310 in which an on-screen text 312 showing the generated utterance information and an illustration image 314 are superimposed on the video Mv is illustrated. In addition, questions or answers such as quizzes based on scripts prepared together with the video contents may be displayed on the on-screen text.

The speaker 5 can output a speech signal corresponding to the utterance information. The speech synthesizer 208 performs text-to-speech (TTS) conversion based on the utterance information that is input from the controller 230, generates a speech output that corresponds to the utterance information of a predetermined setting (specified by language, gender, pitch, tone, speech style, or the like, or by a specific speech model name, etc.), and outputs it through the speaker 5.

The utterance information output section 204, the on-screen text creator 206, or the speech synthesizer 208 are included in a speech information outputter to output utterance information, in the embodiment to be described. In the embodiment to be described, it is assumed that utterance information is output by speech or on-screen text (letters), but this is not limited to this. For example, other output formats such as video output such as sign language expression by CG or expression by movement of a sign-language robot may be used. The details of the utterance information generation processing will be described in the following.

The speech information input section 210 receives an input of speech information from the usage environment via the microphone array 7 and outputs it to the speech recognizer 212. First, the speech information is input to the speech information input section 210 in the form of a digital speech signal obtained by sampling an analog speech signal input to the microphone array 7. At this time, since the microphone array 7 can receive speech input by directions, the speech signal may be generated for each direction. The speech information input section 210 is included in the speech information inputter in the present embodiment.

The speech recognizer 212 applies speech-to-text (STT) conversion to the speech information in a digital speech signal format from the speech information input section 210, converts it into speech information in a text format (e.g., “It's a nice day.”), and outputs it to the controller 230. The speech recognizer 212 may also perform speaker identification based on a voiceprint profile of a specific user U registered in advance, and add speaker information to the speech information in the text format (for example, “It's a nice day, Mr. Yoshida.”). Also, since the speech signal is obtained for each direction as described above, direction information for identifying the direction from which the sound came may be added instead of or together with speaker identification (“right: 60 degrees: It's a nice day, Mr. Yoshida.”). Furthermore, the speech recognizer 212 may perform speech emotion recognition and add attributes representing properties of sounds and voice (tone of voice, and emotions such as, positivity, negativity, anger, elation, enjoyment, boredom, calmness, and sadness) to the speech information in the text format. In addition, in addition to extracting from the speech signal, the attributes related to emotions may be extracted by natural language analysis (sentiment analysis) of the text obtained by the speech recognition. The speech recognizer 212 may also use other speech analysis to detect and add such as the number of breaths from the breath sound. The speech recognizer 212 may also assign an ID to the recognized speech information and add a timestamp (for example, “right: 60 degrees: x/y/2024/10:04.432/It's a nice day, Mr. Yoshida.”).

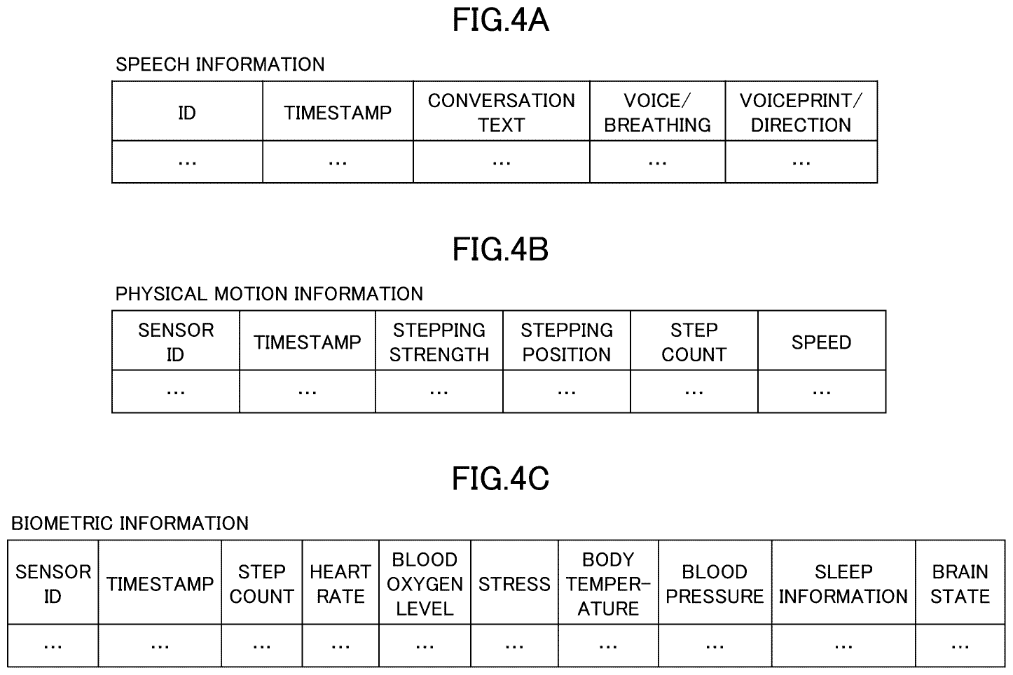

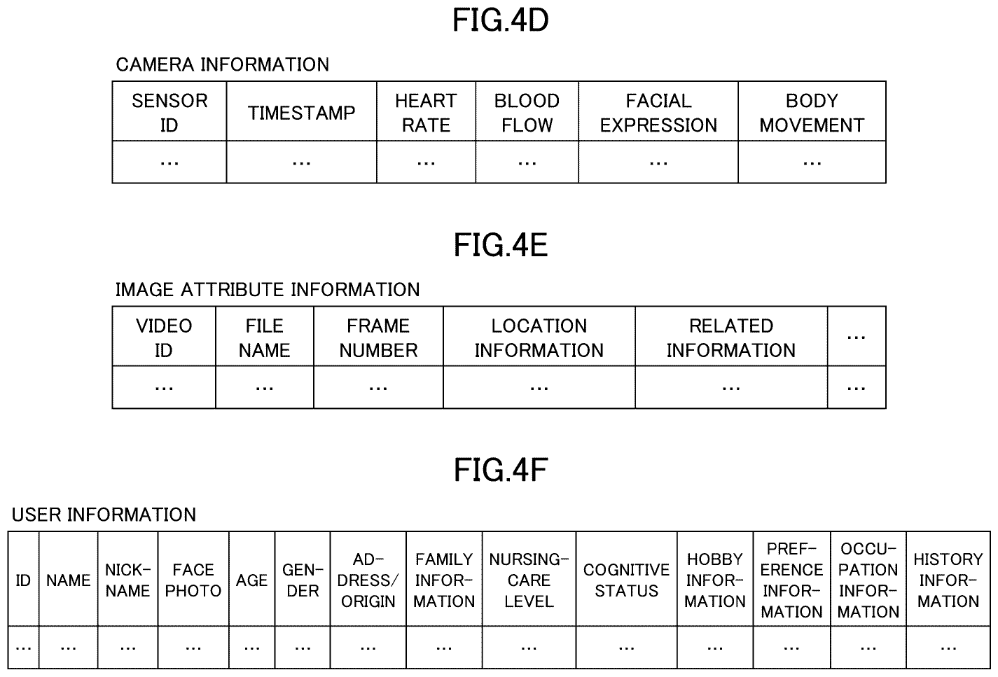

FIGS. 4A to 4F are diagrams illustrating data structures of various types of input information generated by the exercise support system 100 according to one or multiple embodiments of the present disclosure. It should be noted that the data structures as illustrated in FIGS. 4A to 4F are only exemplary, and that data fields are appropriately designed according to specific implementations. FIG. 4A is a diagram illustrating a data structure of speech information. As illustrated in FIG. 4A, the speech information includes an ID field, a timestamp field that holds time information of speech input, a conversation text field that holds conversation contents, a voice/breathing field that holds tone and number of breaths information, and a voiceprint/direction field that holds information for identifying a speaker and direction.

Referring again to FIG. 2, the motion information input section 214 receives motion information related to the walking exercise of the user U via the gait sensor 20 and inputs it to the motion information analysis section 216. Although the motion information also depends on the output format of the gait sensor 20, it may be a raw waveform signal or, as described above, it may be provided in the form of information indicating the occurrence of a predetermined movement (event) such as landing, leaving, stepping, or stopping of the user U. The motion information analysis section 216 converts the motion information in the input format into an output format processed by the controller 230. For example, the motion information analysis section 216 receives a series of inputs of the landing or leaving of the user U's foot, generates motion information (for example, “right/x/y/2024/10:02:30.002” or “left/x/y/2024/10:02:30.450”) in a form indicating the timing of a left/right step with a timestamp (date and time) attached to the motion information, and outputs it to the controller 230.

The motion information analysis section 216 may also add information indicating the stepping strength when the gait sensor 20 can detect it. The motion information analysis section 216 may also use the past motion information together to calculate the number of steps and the speed within a predetermined period and generate motion information in a form in which an average number of steps and average speed are associated with the predetermined period or a predetermined time point. The motion information input section 214 is included in the motion information inputter in the present embodiment, and the motion information analysis section 216 is included in a motion information analyzer in the present embodiment. The motion information analysis section 216 is configured to generate data including at least one of the following information from the motion information: the time length of the walking exercise, the number of steps in a predetermined period, the walking speed at the predetermined time point or the average walking speed in the predetermined period, or the intensity of the walking exercise.

FIG. 4B is a diagram illustrating the data structure of the motion information. As illustrated in FIG. 4B, the motion information includes a sensor ID field that holds an ID for identifying the gait sensor 20, a timestamp field, a stepping strength field that holds information indicating the strength of stepping, a stepping position field that holds information indicating left or right foot or a position on a pad, a step count field, and a speed field.

The above-described gait sensor 20 is a sensor that directly detects the walking exercise of the user, but the method of obtaining the motion information is not particularly limited. For example, the motion recognizer 218 may analyze an image input from the camera 3 (an example of the motion information obtaining device), detect the skeletal structure of the user U, and perform motion analysis to detect a walking exercise. In addition to using the gait sensor 20 and the camera 3, a device including an acceleration sensor or a gyro sensor (the motion information obtaining device) such as of a smartphone may be used to analyze walking from a pattern of acceleration and angular velocity to obtain motion information. In addition, in addition to detecting a state by distinguishing the right and left feet, the motion information may include count-up information (for example, “1 time: x/y/2024/10:02:30.002” or “1 time: x/y/2024/10:02:30.450”) from a device that counts steps without distinguishing the right and left feet, such as a pedometer (registered trademark).

The facial expression input section 219 detects a face area of a person from the captured image of the camera 3 (an example of the reaction information obtaining device), recognizes the facial expression of the person from the face image, and transmits the facial expression information to the controller 230.

The user identification section 220 detects the face area of a person from the captured image of the camera 3, identifies a specific user U from the face image, and transmits the information to the controller 230. The face image for identifying each user U is stored in the user information storing section 246. The user identification section 220 is included in a user identifier according to the present embodiment.

The biometric information input section 222 receives input of biometric information (heart rate, blood oxygen level, activity level, etc., of the wearer) related to the body of the user U from the biosensor 23. The biometric information input section 222 is included in a biometric information inputter of the present embodiment.

The biometric information analyzer 224 analyzes the biometric information, adds timestamps, user information, and the like to the biometric information, and transmits it to the controller 230. Various types of the biosensor 23 can be exemplified. For example, in addition to the smartwatch and pedometer (registered trademark) described above, an activity meter (activity tracker), a sleep meter (sleep tracker), a blood pressure meter, a small brain-activity sensor, and a camera such as a smartphone can be exemplified.

FIG. 4C is a diagram illustrating the data structure of biometric information. As illustrated in FIG. 4C, the biometric information includes a sensor ID field for identifying the biosensor 23, a timestamp field, a step count field, a heart rate field, a blood oxygen level field, a stress value field, a body temperature field, a blood pressure field, a sleep information field, and a brain state field.

FIG. 4D is a diagram illustrating a data structure of the biometric information that can be obtained by a camera. For example, there is a known technology for estimating the heart rate and a respiration rate by photographing a face with a camera. In addition, as described above, it is possible to recognize facial expressions (emotions) and to detect body movements from a face image. As illustrated in FIG. 4D, the biometric information includes a sensor ID field for identifying the camera, a timestamp field, a heart rate field, a blood flow field, a facial expression field, and a body movement field.

Based on the input from the controller 230, the notification section 228 notifies a previously registered contact (by e-mail, messaging system, social network system, external care system, etc.,) of various information. The notification section 228 is called in response to the controller 230 detecting an abnormality based on biometric information, such as the heart rate measured by the biosensor 23 exceeding the upper limit, or detecting an abnormality based on speech information, such as the speech information output from the speech recognizer 212 containing a word for requesting SOS. The notification section 228 is included in a notifier according to the present embodiment.

The video storage 240 is configured to store a plurality of video contents to be played back when performing a walking exercise, and in addition, store scripts and attribute information in association with the video contents. The scripts are on-screen texts to be displayed on the screen in accordance with the progress of the video, narrations to be output in speech, and data to describe operation control of external devices such as lighting devices and air conditioners during video playback. In response to the selection of the video contents to be played back via the operation section 202, the controller 230 reads the video data from the video storage 240 and transfers it to the video playback section 226 to play back the video.

The video analyzer 242 analyzes the video data of the video contents in advance or in real time, recognizes objects (things) included in the image by image analysis such as named entity recognition (NER), and may add a tag for identifying the objects (things, places, buildings, plants and animals, food, people, pictures, signs, etc.), and may add descriptive information (such as “a person is walking” or “a dog is barking”) of the contents of each frame by video captioning and the like. These tags and texts are stored in the video storage 240 as image attribute information associated with the video contents, a specific frame in the video contents, and time information. The image attribute information added to the video contents may be automatically added by analysis by the video analyzer 242, or may be manually added. In addition, the video contents may be added with geographic coordinates of the shooting location.

In the video taken while strolling in a sightseeing area, the video analyzer 242 may estimate the walking speed of the photographer from the moving speed of the scenery in the image and add speed information to each frame such that the video is a video at a standard speed. By using such speed information or time information, in playback control of the video, for example, in a frame of 0 speed such as a scene where the photographer stops, the playback control according to the pace of the walking exercise can be temporarily cancelled or the playback speed can be corrected (for example, correction for matching the moving speed of the photographer between a plurality of sections, in a video in which the moving speed of the photographer differs between the sections).

FIG. 4E is a diagram illustrating a data structure of the image attribute information added to video data by the video analyzer 242 or manually. As illustrated in FIG. 4E, the image attribute information includes a video ID field for identifying a video, a file name field for holding a file name, a frame number field for identifying a frame number, a location information field for indicating a position or a location (place name such as Asakusa, or geographical (global positioning system: GPS) coordinates indicating the location) associated with the frame number, and a related information field for holding related information (specialties, celebrities, history, topics, architecture, etc.) associated with the position. The frame number field, the location information field, and the related information field may be provided for each frame or for each group including a plurality of frames (for example, each scene (section) when the entire video is divided into a plurality of scenes (sections)).

The conversation example storage 244 stores conversation examples prepared by a developer in advance as well as examples collected from actual conversations between a computer and the user U (automatic inquiry to the user U and response from the user U, inquiry from the user U and automatic response to the user U) achieved by the controller 230.

The user information storing section 246 stores user information (name, gender, age, background, residency, work history, travel history, hobbies, sports experiences, and preference information) associated with each user for each user. The user information storing section 246 also stores a face image referenced by the user identification section 220 in association with the user and provides the face image to the user identification section 220. In addition, the user information storing section 246 stores, in association with the user, the contents of a conversation performed during a walking exercise session performed by the user in the past and information (average walking pace, number of steps, etc., in past walking exercises) based on motion information from the gait sensor 20. The user information storing section 246 is included in a user information storage, an information storage, or both of these in the present embodiment.

FIG. 4F is a diagram illustrating a data structure of user information stored in the user information storing section 246. As illustrated in FIG. 4F, the user information includes an ID field, a name field, a nickname field, a face photo field, an age field, a gender field, an address/origin field, a family information field, a nursing-care level field, a cognitive status field, a hobby information field, a preference information field, an occupation information field, and a history information field. It should be noted that the data structure with specific fields as illustrated in FIG. 4F is an example and is designed according to a specific implementation.

In the embodiment to be described, information stored in the video storage 240, the conversation example storage 244, and the user information storing section 246 is mainly used, but external information may also be used. For example, the exercise support system 100 may be provided with a module that cooperates with the outside for importing external data or exporting data, or using search results of an external search engine.

Hereinafter, more specific functions of the controller 230 will be described, including generation processing of utterance information by the controller 230.

More specifically, the controller 230 includes an utterance information generator 232, a video controller 234, and a report information generator 236. The utterance information generator 232 generates a question to the user and a response to the question from the user by using input information (speech information, motion information, image attribute information, biometric information, and facial expression information) input from each input section to the controller 230, the conversation examples stored in advance, the examples of conversation in the past, the user information, and the like. The video controller 234 generates a playback speed of the video contents, a stop instruction, a restart instruction, and the like based on speech information, motion information, image attribute information, biometric information, and facial expression information that are input from the input sections to the controller 230, and controls playback of the video in cooperation with the video playback section 226. The utterance information generator 232

can generate utterance information related to the exercise support for the user by using reaction information related to a reaction of the user and motion information that is input to the motion information input section 214 and generated by the motion information analysis section 216, as inputs. In the embodiment to be described, the reaction information related to a reaction of the user is speech information that is input by the user in response to a request to the user from the exercise support system 100, such as the utterance information generated and output in the previous time by the utterance information generator 232. More specifically, the reaction information related to a reaction of the user is speech information in a text format obtained by inputting the utterance of the user into the speech information input section 210 and being recognized as speech information and converted into a text format by the speech recognizer 212.

In addition, the utterance information related to the exercise support for the user is utterance information including a content of encouraging the user about physical exercise. The utterance information may include utterances for promoting exercise (for example, “Would you like to pick up the pace a little bit?”, “Let's keep it up for a few more minutes.”, etc.), utterances for suppressing exercise (for example, “Let's slow down a little bit.” or “Take it easy.”), and utterances for stopping exercise (for example, “Let's stop for today.”).

For example, when the speech information indicates silence and the motion information indicates a decrease in the walking pace, the utterance information generator 232 generates utterance information (for example, “The pace of walking has slowed down. Would you like to take a break?”) to encourage a break. In addition to the speech information and the motion information, the utterance information generator 232 can generate utterance information by using image attribute information output by the video playback section 226 as an input. For example, when the speech information indicates silence, the motion information indicates a decrease in the walking pace, and the image attribute information indicates a “viewing point”, the utterance information (for example, “There is a nice view. Shall we take a break here?”) to encourage a break is generated.

As described above, location information is attached to the image attribute information, and related information may be attached to the location information. The utterance information generator 232 can generate utterance information based on the location information and related information. For example, when the speech information indicates that a conversation continues, the motion information indicates that the walking pace is within a normal range, the location information indicates that the scene of “Asakusa” is displayed, and the related information “Kaminarimon” is attached to the location information “Asakusa”, the utterance information (for example, “Everyone is still energetic. Speaking of Asakusa, Kaminarimon is famous. Have you ever been there?”) is generated by using this information as inputs. The generated utterance information is sent to the utterance information output section 204 and is output in on-screen text, speech, or both formats.

The utterance information generator 232 may further generate utterance information by using the user information stored in the user information storing section 246 corresponding to the identified specific user among a plurality of users as inputs. For example, when the speech information indicates the user with the largest number of utterances and the user information of the user indicates that the downtown area is the place of origin, the utterance information generator 232 generates utterance information (for example, “You are from downtown, aren't you? Do you recognize the scenery around here?”).

The utterance information generator 232 can also generate utterance information (for example, when the motion information indicates that a recommended range has been exceeded, “You're excited. You can walk a little more slowly.” or the like) that leads the user to suppress the amount of exercise based on at least one of the reaction information (speech information), motion information, or biometric information. The utterance information generator 232 may also generate utterance information (for example, “Isn't your heart beating a little fast? Shall we take a break?” when the biometric information indicates that the heart rate has exceeded the recommended range) that leads the user to stop the exercise based on at least one of the information. The utterance information generator 232 can also generate utterance information (for example, when the motion information indicates that the activity level of the walking exercise of a plurality of users has fallen below a certain level, “Do you like shopping?” based on the relevant information “Nakamise Dori” related to the location “Asakusa” and the relevant information “shopping street” associated with other relevant information based on general knowledge) that leads the user to change the topic based on at least one of the information. The utterance information generator 232 may also generate utterance information (for example, “You're walking more briskly than usual today. Did anything good happen?” based on information on a past walking pace of a particular user) by using past information stored in the user information storing section 246 as an input. It should be noted that the generation method of utterance information described here is only an example and is not limited.

It should be noted that in the described embodiment, the reaction information related to a reaction of the user is speech information of the user's utterance that is input (for example, within a predetermined period) to the speech information input section 210 in response to the request to the user and recognized. However, the reaction information related to a reaction of the user is not limited thereto. In other embodiments, the facial expression information may be the expression information identified from the face image information indicating the facial expression of the user, which is input (for example, within a predetermined period) to the facial expression input section 219 in response to an approach to the user, or the biometric information related to the user's body, which is input (for example, within a predetermined period) to the biometric information input section 222 in response to a request to the user. Hereinafter, the description will be continued assuming that the reaction information is speech information.

The video controller 234 controls the video (image information) to be output to the video playback section 226 to be changed based on the input motion information. Here, changing the video may mean changing the speed at which the video is played back. For example, when the motion information is an average walking pace of a plurality of users (or a trimmed average value obtained by excluding the maximum and minimum values (or the upper limit or the lower limit of a predetermined ratio)), the playback speed can be adjusted such that the average value matches with the progress speed of the video. Changing the video may also mean changing the contents of the image information or inserting one or both of the on-screen text and an insertion image into the image. The video controller 234 is included in the image controller in the present embodiment. It should be noted that such adjustment of the playback speed of the video, changing the contents of the image information, and inserting one or both of the on-screen text and the insertion image into the image may also fall under the above-mentioned approach to the user.

The report information generator 236 compares the past information or predefined reference information stored in the user information storing section 246 with the input current information, generates report information based on a comparison result, and calls the notification section 228 to transmit the report information to a predetermined notification destination. The report information generator 236 is included in the report information generator in the present embodiment. The generation of report information by the report information generator 236 will be described in detail in the following.

The notification destination by the notification section 228 is an external care system, an e-mail address, an account of a messaging system, or an account of a social network service (SNS), and is stored, for example, in the user information storing section 246. The notification section 228 transmits information to a predetermined destination stored in the user information storing section 246 via a network 25.

The controller 230 can also store dialogue information as specific user information, record the state of engagement in the walking exercise (number of steps, average walking pace, walking time, etc.), or record newly extracted user information (for example, badminton is added to the sports experience in the user information based on the dialogue that the user used to play badminton) in accordance with the dialogue with the user during the walking exercise.

The utterance information generator 232 may generate utterance information by using a machine learning model 260 that outputs utterance information with motion information and reaction information (speech information) as inputs, or may generate utterance information based on branching logic that maps utterance information conditionally on the motion information and reaction information (speech information). The video controller 234 can also change the playback speed of the motion by the machine learning model 260 or the branching logic.

Hereinafter, processing of generating utterance information by using the machine learning model 260 to which motion information and reaction information (speech information) are input will be described more specifically. The functional block 200 as illustrated in FIG. 2 further includes a training section 248, a training data storage 250, and a machine learning model 260.

The training data storage 250 stores training data for supervised learning of the machine learning model 260. The training data associates output data (label, value, or text) that is ground truth with predetermined input data. When learning a conversation, multiple sets of questions and responses to the questions are prepared as the training data. The training section 248 updates the parameters of the machine learning model 260 by applying a predetermined machine learning algorithm. The training section 248 may construct a machine learning model to be used from scratch, or may prepare a machine learning model 260 to be used by re-learning a pre-learned model.

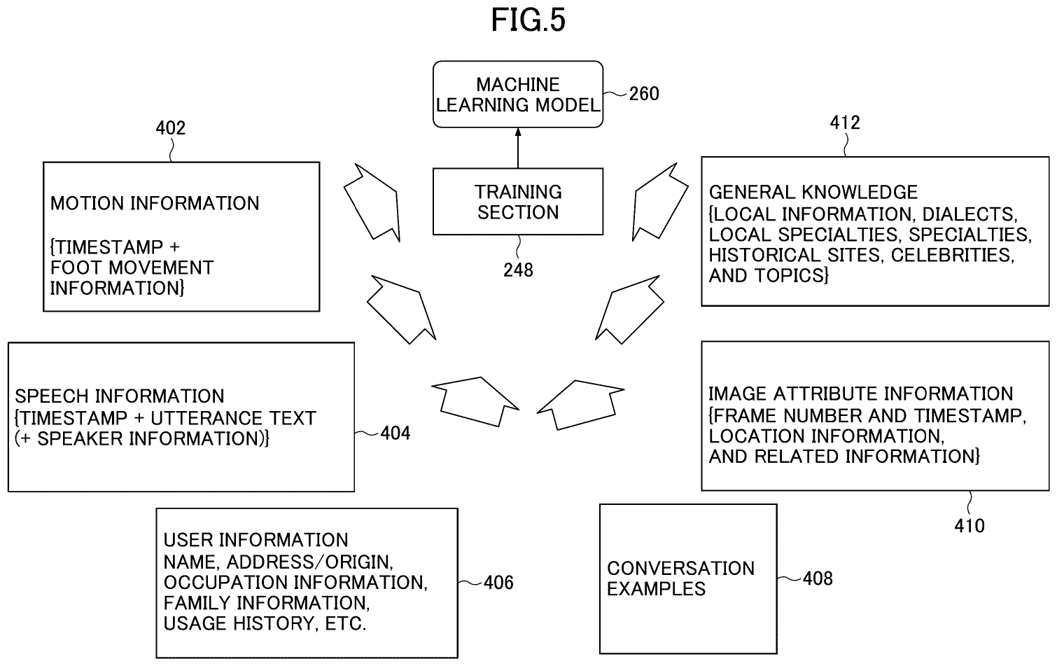

FIG. 5 is a schematic diagram illustrating a learning process of the machine learning model 260 in the exercise support system 100 according to one or multiple embodiments of the present disclosure.

As the training data for training the machine learning model 260, (a) pre-training data prepared in advance on the developer's side, and (b) historical training data created based on the history data generated during the use of the exercise support system 100 and obtained with permission from the relevant persons including the user and the manager of the facility within the scope of a predetermined use purpose are assumed. As the (a) pre-training data, for example, (a-1) training data obtained by manually describing a conversation during walking training, which is a model case, and (a-2) training data obtained by recording an interaction that a participant actually engaged in while watching a video and walking exercise in a test environment (for example, in a state where a conversation function is disabled) of the exercise support system 100 are assumed. The training data in (a-1) includes conversation examples, and in the training data in (a-2) and (b), in addition to the conversation examples, motion information, biometric information, and image attribute information of a video to be viewed can be obtained. The (b) historical training data may be used for re-learning in the exercise support system 100 in a form limited to use within a specific facility, or it may be used for re-learning a shared model of the exercise support system 100 after obtaining permission from the relevant persons including the purpose of using the training for the shared model with other facilities.

The training data in (a-2) can be obtained as follows. For example, a caregiver or a nursing staff member as a participant on an operator O side, and a monitored elderly person as a participant on a user side, watch predetermined video contents in a test environment, and speech information, motion information, and biometric information are collected during conversation while performing walking exercises. The speech information is recorded separately for the participant on an operator O side and the participant on the user side. Video data to be viewed is provided with image attribute information associated with frames by video analysis in advance, and also, the attribute information that is corrected and to be added is manually prepared as required. User information can also be prepared for the user side participant.

As illustrated in FIG. 5, motion information 402, speech information 404, user information 406, conversation examples 408 such as (a-1), image attribute information 410, and general knowledge 412 are prepared in a test environment. Here, the general knowledge 412 is information associated with local information, dialects, regional products, local specialties, historical sites, celebrities, and topics in a predetermined topology. Time information is linked to the motion information 402, the speech information 404, the user information 406, the conversation examples 408, and the image attribute information 410 by timestamps, frame numbers, and the like. Timestamps indicating time are assigned to the motion information 402 and the speech information 404. Frame numbers are assigned to the image attribute information, and the frame numbers can be converted to the same time as the time indicated by the timestamps based on the video playback start time and video playback speed.

Since time information is associated with these pieces of information, speech information, motion information, and image information are ordered as a whole in the order of occurrence. By using these pieces of information associated with the time information, a collection of training data of the machine learning model 260 can be generated. The collection of training data is given to the training section 248, and the training section 248 prepares the machine learning model 260 based on the given information. The training section 248 may prepare a dedicated machine learning model for each video content, for example, but more preferably, it prepares a machine learning model that can be applied to various video contents in general.

Through the learning process, examples of conversations such as what the caregiver said in the video scene, how the user responded, or what the user talked to and how the caregiver responded to in the conversation are learned. Since the training data also includes data based on motion information and image attribute information that matches with the display contents of the video, it is possible to learn what kind of conversation was made in which video, according to the participant's reaction (increase in walking motion, conversation frequency, etc.).

As the machine learning model 260, large-scale language models (LLM) such as Generative Pre-trained Transformer (GPT)-2, GPT-3, GPT-3.5, GPT-4, GPT-40, Bidirectional Encoder Representations from Transformers (BERT), XLNET, ChatGPT, and the like can be used, although not specifically limited. The LLM can be learned through (I) prior learning in which large-scale parameter learning is performed by using a large corpus, (II) supervised fine tuning (SFT) in which supervised learning is performed by using instructions, and (III) reinforcement learning with human feedback (Reinforcement Learning from Human Feedback: RLHF) in which the model is executed for a large number of instructions and humans feedback the superiority to the output. Conveniently, for example, the training section 248 can (II) relearn the machine learning model by the SFT as the instruction by using the conversation examples described above.

Learning methods (I) to (III) described above are learning processes that involve updating the parameters of the LLM itself, but when using the LLM, in-context learning can also be performed. The in-context learning is a so-called prompt statement in which instructions for a specific task are given to the LLM in advance to obtain an optimal output. This is different from learning in the usual sense that involves updating the parameters of the model. However, by providing prior information, the LLM can be optimized for a specific task.

In existing LLMs such as GPT, although GPT-4 permits images as inputs, text is the main input and output, and multiple LLMs are exchanged in a conversational manner. In contrast to this, in the exercise support system 100 in one or multiple embodiments of the present disclosure, motion information, biometric information, and image attribute information are used as input information. In order to use motion information and biometric information as inputs to the LLM, a definition of the format of the input information can be given in the prompt statement at the beginning as necessary, and conversational motion information and biometric information can be generated based on the motion information and the biometric information.

For example, the motion information is given as a timestamp and information such as a left or right step, and a walking pace is calculated from a series of pieces of motion information, and a text sentence (for example, “The walking pace is now 70 steps per minute.”) including the motion information is generated together with a normal conversation sentence or as a separate conversation sentence when the walking pace satisfies a predetermined condition (for example, the walking pace exceeds a threshold value, a change amount in walking pace per unit time exceeds a threshold value, etc.). Since biometric information is also given as information such as a timestamp and a heart rate, a text sentence (for example, “My heart rate has increased to 130 beats per minute.”) containing biometric information is generated in conjunction with a normal conversation sentence or as a separate conversation sentence when a predetermined condition (for example, the heart rate exceeds a threshold value, a heart rate change amount per unit time exceeds a threshold value, etc.) is satisfied. Also, regarding image attribute information, a text sentence (for example, as a result of analyzing the image of the video, utterance information “I saw Kaminarimon.” is recognized) containing information describing a video is generated in conjunction with a normal conversation sentence or as a separate conversation sentence at a corresponding timing such as a change of a scene. By inserting such a text sentence based on motion information or image attribute information into a conversation, it is possible to learn speech generation logic and generate utterance information in consideration of motion information, biometric information, and image attribute information.

In addition, for main conversation examples, user information, and the like, it is possible to generate utterance information in consideration of this prior information by performing the in-context learning by adding information to a prompt sentence before starting a walking exercise.

Although various components are illustrated and explained in FIG. 2, these are only examples, and some components as illustrated in FIG. 2 may be omitted, or other components not illustrated in FIG. 2 may be added. In the embodiments as illustrated in FIGS. 1 and 2, the information processing apparatus 1 is described assuming that the information processing apparatus 1 is responsible for most of the functional blocks, as indicated by the dashed rectangle 1, for convenience. However, since a machine learning model such as the LLM consumes large resources, it may be provided outside the information processing apparatus 1. For example, as indicated by a dotted rectangle 10 in FIG. 2, the training section 248, the training data storage 250, and the machine learning model 260 may be provided outside the information processing apparatus 1. In this case, the information processing apparatus 1 has a communication function to communicate with an external application programming interface (API) on an LLM side, and by receiving API calls and responses, it is possible to outsource most of the computational load to outside the information processing apparatus 1, thereby reducing the hardware resource requirements required for the information processing apparatus 1.

Furthermore, components such as the speech synthesizer 208, the speech recognizer 212, the motion recognizer 218, and the video analyzer 242 may also be outsourced to outside the information processing apparatus 1 as communication via an API. In another embodiment, the information processing apparatus 1 may be implemented by providing only the minimum components necessary for communication with the gait sensor 20 and the display 4, and communicating other components with an external server device or a server application (including a case where the server application further communicates with the LLM via the API) deployed on the cloud infrastructure.

By using a machine learning model such as a large-scale language model, it is possible to efficiently construct an interaction model, and it is easy to update and extend the interaction model.

In the above description, it is assumed that the video content is played back along with the walking exercise, but it is not necessary to necessarily play back the video content. For example, in another embodiment, the walking exercise may be performed while simply viewing a TV program while the TV program is displayed on another monitor. In this case, image attribute information corresponding to the video contents is not provided, but when the audio of the TV program can be collected and processed by the microphone array 7, the contents of the TV program can be substantially incorporated into the conversation.

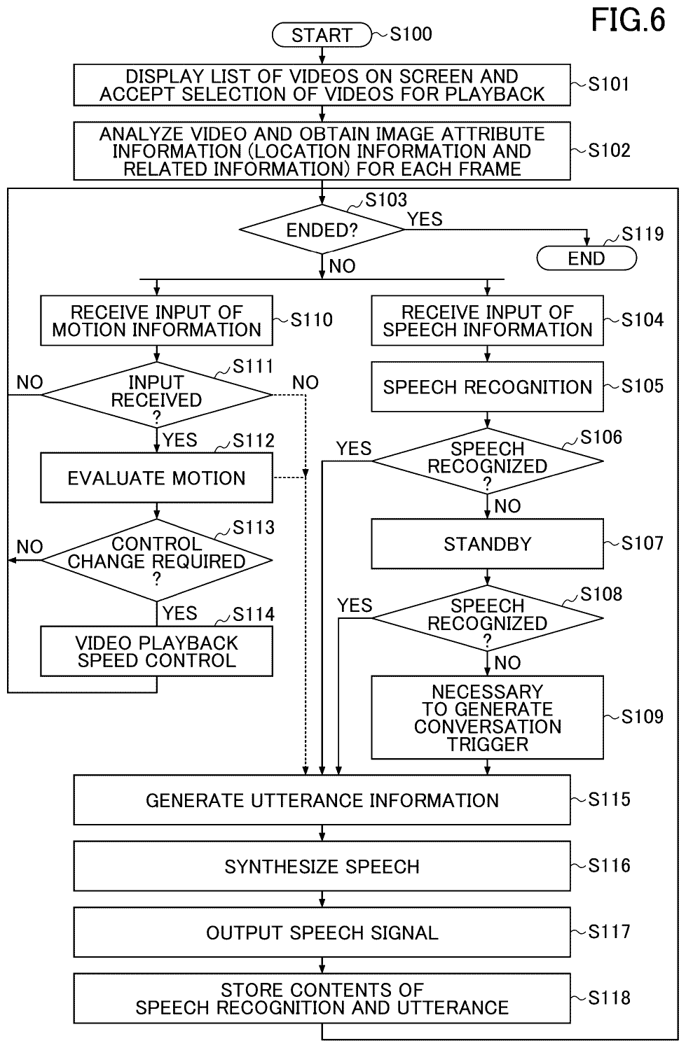

Hereinafter, with reference to FIG. 6, the above-described walking exercise support processing with automatic interaction will be described more specifically. FIG. 6 is a flowchart illustrating the walking exercise support processing executed by the information processing apparatus 1 according to one or multiple embodiments of the present disclosure. The processing as illustrated in FIG. 6 starts at step S100, for example, in response to the system startup.

In step S101, the information processing apparatus 1 displays a list of videos on the screen by the operation section 202 and accepts the selection of videos to be played back. In step S101, the content selection screen 300 as illustrated in FIG. 3A is displayed, and the video contents are selected on the screen 300. It is assumed that the registration of the participating users, the association of each participating user with the gait sensor 20, and the voiceprint profiles of the participating users are completed prior to the start of the walking exercise. Furthermore, from the start to the end of the walking exercise is referred to as a session.

In step S102, the information processing apparatus 1 analyzes the video data of the video contents read from the video storage 240 by the video analyzer 242, and obtains location information and related information for each frame (frame group).

In step S103, the information processing apparatus 1 determines whether or not the walking exercise session using the video content has ended by the controller 230. When it is determined in step S103 that the walking exercise session has not ended (NO), the processing proceeds to step S104 and step S110, and the processing for the speech information as illustrated in steps S104 to S109 and the processing for the motion information as illustrated in steps S110 to S114 are executed in parallel.

In step S104, the information processing apparatus 1 receives input of speech information by the speech information input section 210. In step S105, the information processing apparatus 1 converts the speech information in the form of a speech signal into speech information in the form of a text by the speech recognizer 212. A timestamp and user identification information by speaker identification are applied as appropriate. In step S106, the information processing apparatus 1 determines whether or not a meaningful text is generated by the speech recognition.

When it is determined in step S106 that a meaningful text has been generated and speech recognition has been performed (YES), the processing proceeds to step S115. In contrast to this, when it is determined in step S106 that a meaningful text has not been generated and speech recognition has not been performed (NO), the processing proceeds to step S107, and after waiting temporarily, the processing proceeds to step S108. Here, it is intended to exclude a meaningless text generated due to environmental noise or failure of speech recognition. Thus, the information processing apparatus 1 waits for another utterance from the user. In step S108, the information processing apparatus 1 performs speech recognition again and determines whether or not a meaningful text has been generated. When it is determined in step S108 that a meaningful text has been generated and speech recognition has been performed (YES), the processing proceeds to step S115.

In contrast to this, when it is determined in step S108 that still no meaningful text has been generated and speech recognition has not been performed, the processing proceeds to step S109. This is because when there is no speech for a certain period of time or longer, the spontaneous speech of the user cannot be expected even when the information processing apparatus 1 waits any longer, and it is effective to encourage the user to actively discuss about a topic or request speech. In step S109, the information processing apparatus 1 records that it is necessary to generate a conversation trigger, transmits the information to the utterance information generator 232, and proceeds the processing to step S115.

In step S110, the information processing apparatus 1 receives input of motion information from the motion information input section 214. In step S111, the information processing apparatus 1 determines whether or not motion information has been input. When it is determined in step S111 that there is no input of motion information (NO), the information processing apparatus 1 records that there is no input of motion information, transmits the information to the utterance information generator 232, terminates the processing related to motion information, and returns to step S103.

In contrast to this, when it is determined in step S111 that there is input of motion information (YES), the processing proceeds to step S112. In step S112, the information processing apparatus 1 analyzes the input motion information, including motion information that was input in the past, by the motion information analysis section 216, and evaluates the motion. For example, the walking pace is calculated, and whether or not the walking pace is within an appropriate range is evaluated. For example, the evaluation result indicating that the walking pace is within the predefined lower limit value and upper limit value, the walking pace is less than the lower limit value, or the walking pace exceeds the upper limit value is obtained. The evaluation result of the motion is sent to the utterance information generator 232. In this way, the motion information analysis section 216 generates evaluation information of physical exercise, and the utterance information generator 232 can generate utterance information by using the generated evaluation information as motion information. In step S113, the information processing apparatus 1 determines whether or not a control change is necessary by the video controller 234. When it is determined in step S113 that no control change is necessary (NO), the processing concerning the motion information ends and the processing returns to step S103.

In contrast to this, when it is determined in step S113 that a control change is necessary (YES), the processing proceeds to step S114. In step S114, the information processing apparatus 1 changes the video progress speed by the video controller 234, the processing concerning the motion information ends, and the processing returns to step S103.

When the processing for the speech information as illustrated in steps S104 to S109 and the processing for the motion information as illustrated in steps 110 to S114 end, the processing proceeds to step S115.

In step S115, the information processing apparatus 1 generates utterance information based on the information obtained so far by the utterance information generator 232. Regarding speech information, when speech recognition is successful in step S106 or step S108, the utterance information generator 232 receives the speech information in recognized text format, and when the speech recognition again is not successful (NO) in step S108, the utterance information generator 232 receives information that generation of a conversation trigger is necessary, in step S109. Regarding the motion information, when it is determined that there is no motion input in step S111, the utterance information generator 232 receives information that there is no motion input, and when it is determined that there is motion input in step S111, the utterance information generator 232 receives a motion evaluation result, in step S112.

In step S115, the utterance information generator 232 generates utterance information based on the machine learning model 260 or the branching logic based on the obtained processing result of the speech information (information that a text or a trigger is necessary) and the processing result of the motion information (information that there is no walking pace or motion input). The generation of the utterance information is as described above, but when the LLM is used, a request statement to the LLM is created based on the processing result of the speech information and the processing result of the motion information, and transmitted to the LLM via the API, for example, to obtain a response statement. According to the configuration of the LLM, the request statement includes information about past interaction.

When a trigger is necessary, there is no text statement of speech information, and when there is evaluation information of the motion information, for example, a text statement including the evaluation information (“Now walking at an average pace of 70 steps per minute.” etc.) is included in the request and transmitted to the LLM. Then, a response statement such as “You are walking at a good pace.” is obtained, and this is used as speech information. Or, although omitted in the flowchart, when there is biometric information (heart rate, etc.), a text statement including biometric information (for example, “The heart rate is 110 beats per minute.”) is included in the request and transmitted to the LLM. Then, an answer such as “Moderate exercise. You're in good shape.” is obtained, which is used as utterance information. Alternatively, when there is location information and related information associated with the frame of the currently displayed video, a text (for example, “I can see Kaminarimon.” “I can see a souvenir shop.”) including the location information and related information is included in the request and transmitted to the LLM. Then, an answer such as “The official name of Kaminarimon is ‘Furaijinmon’.” is obtained, which is used as utterance information. The processing of obtaining a text from attribute information as a keyword such as “Kaminarimon” or “Nakamise Dori” may be separately generated by cooperation with the LLM, or a text may be generated by the branching logic from the keyword. In addition, multiple pieces of related information may be associated with each other. In this case, the contents described in the user information of the participating user and its relevance may be evaluated, and the most relevant content may be selected.

When the utterance information is generated by the utterance information generator 232 in step S115, the processing proceeds to step S116. In step S116, the utterance information in the text format is converted by TTS, the utterance information in a speech signal format is generated, and in step S117, a speech signal is output to the speaker 5. In step S118, the information processing apparatus 1 stores the contents of speech recognition and utterance information by associating them with the user by the controller 230 when the speaker is identified, or when the speaker is not identified, stores the contents of speech recognition and utterance information as a general conversation example, and the processing returns to step S103. When the speech signal is generated in step S116 based on the utterance information generated in step S115, and the speech signal is output in step S117, the speech signal corresponds to an approach to the user from the exercise support system 100. Then, based on the output of the speech signal, in the next cycle, the speech information obtained in steps S104 to S108 is the reaction information related to a reaction of the user (including a case where there is no speech information and no response).

In step S103, when it is determined that the walking exercise session has ended (YES), such as when the playback of the video ends or when the operator O instructs the user to end the walking exercise session with the remote controller 6, the processing proceeds to step S119, and the present processing ends.

When the walking exercise session ends, for example, as illustrated in FIG. 3D, an end screen 320 showing the execution result of the current walking exercise session is displayed. In the end screen 320, result information 322 such as the number of steps of each user U is displayed in a tile-like manner, and a button 324 for returning to the content selection screen is displayed. When the button 324 is selected, the screen returns to the content selection screen 300 as illustrated in FIG. 3A.

When the walking exercise session ends, the user information storing section 246 may record usage history information in association with each user who participated in the walking exercise session. As the usage history information, the used video contents, the number of steps, average, and walking pace in the session (statistic values such as maximum, minimum, and average), and information extracted from conversations in the session (hobbies, experiences, friends, acquaintances, hometown, occupation, sports, other experiences, interests, etc.) are listed.

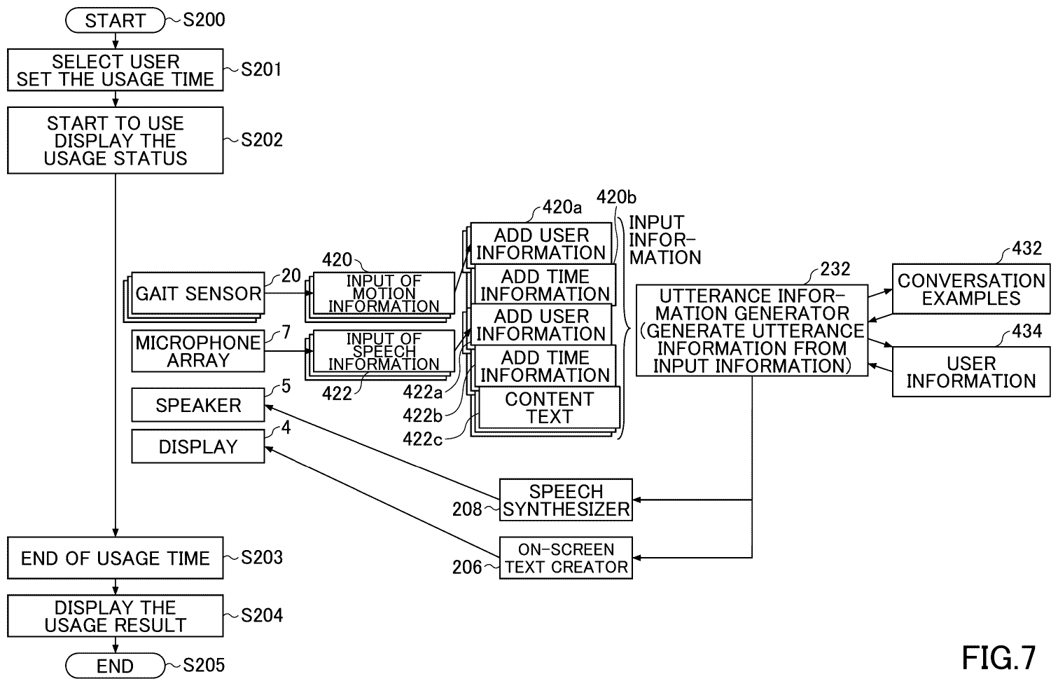

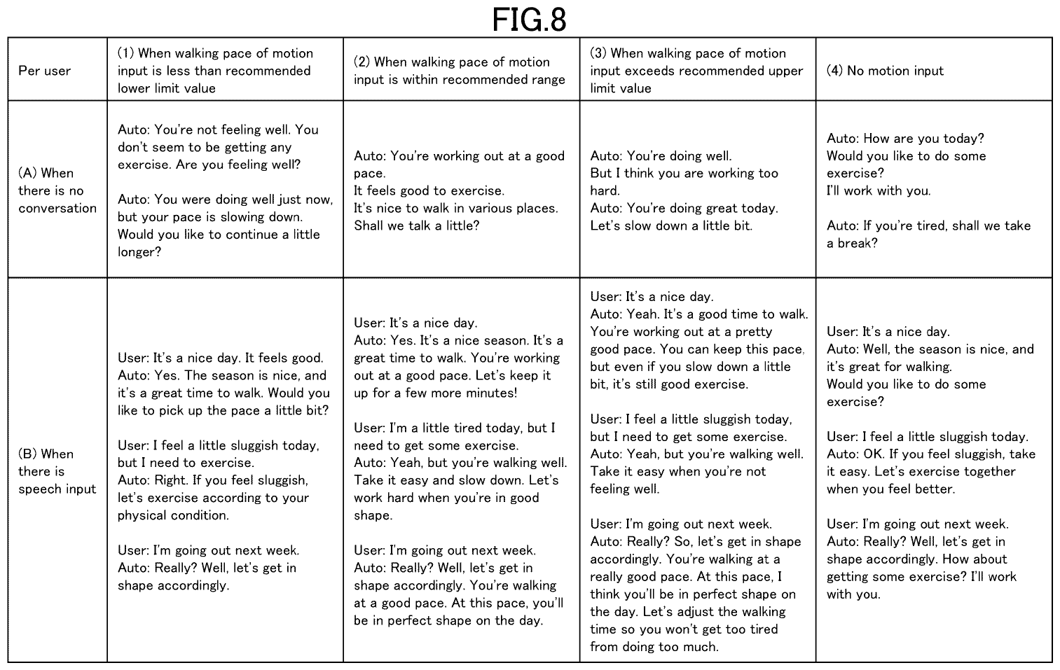

Hereinafter, with reference to FIGS. 7 and 8, processing for generating utterance information based on motion information and speech information will be described more specifically. FIG. 7 is a diagram illustrating the utterance information generation processing based on motion information and speech information executed by the information processing apparatus 1 according to the first embodiment of the present disclosure. Note that in FIG. 7, no video is played back, and for example, the user U performs a walking exercise while watching a general TV program on a television.

FIG. 7 is a flowchart illustrating a series of flows from the start (S200) to the end (S205) of the walking exercise support processing, and data flows between blocks are illustrated next to the flowchart. As illustrated in the flowchart of FIG. 7, the walking exercise support processing starts from step S200, and in step S201, the user selection is accepted. The user registration may be performed manually by using the remote controller 6 or automatically by using the camera 3 based on a face image. In this case, specification of the usage time, registration of the user, and registration of the association between the user and the gait sensor 20 are performed by the remote controller 6 or the like. When the registration is completed, in step S202, the use is started, and a usage status is displayed (on a user screen as illustrated in FIG. 3B except for the video Mv). In step S203, the use ends according to the lapse of the usage time. In step S204, a usage result as illustrated in FIG. 3D is displayed, and in step S205, the walking exercise support processing ends and waits for another instruction.

In the flowchart as illustrated in FIG. 7, from the start of the use in step S202 to the end of the usage time in step S203, motion information 420 is input from each of the one or more gait sensors and speech information 422 about a plurality of directions is input from the microphone array 7 at any time. The motion information analysis section 216 adds user information 420a and time information 420b to the motion information 420. The speech recognizer 212 adds user information 422a, time information 422b, and content text 422c to the speech information 422. This input information is sent to the utterance information generator 232.

The utterance information generator 232 generates utterance information based on the input information. When there is an utterance from the user, the speech information in the text format is input to generate a conversation, and when there is a motion input, the evaluation information of the exercise is input to generate a conversation. In this case, the conversation examples 432 and the user information 434 may be used.

FIG. 8 is a table illustrating conversation examples generated based on motion information and speech information executed by the information processing apparatus 1 according to the first embodiment of the present disclosure. In the table illustrated in FIG. 8, columns indicate states of motion input, and four states are exemplified: (1) when the walking pace of motion input is less than the recommended lower limit value, (2) when the walking pace of motion input is within the recommended range, (3) when the walking pace of motion input exceeds the recommended upper limit value, and (4) when there is no motion input. In the table illustrated in FIG. 8, rows indicate states of input of speech information, and two states are exemplified: (A) when there is no conversation, and (B) when there is speech input.

The conversation (A) when there is no conversation includes only an utterance from the system, as indicated by “Auto”, and this utterance corresponds to a case where it is recorded in step S109 of FIG. 6 that it is necessary to generate a conversation trigger, and it is generated based on the information in step S115. This conversation may be generated by the machine learning model 260 such as the LLM, or a collection of one or more examples of utterance information (432 in FIG. 7) may be defined in advance in association with the state of motion input as illustrated in the table in FIG. 8, and it may be selected probabilistically from the examples of utterance information.

In addition, (4) when there is no motion input, it corresponds to a case where there is no motion input is communicated in step S111 and the utterance information is generated in step S115. Furthermore, (1) when the walking pace of the motion input is less than the recommended lower limit value, (2) when it is within the recommended range, and (3) when it exceeds the recommended upper limit value, it corresponds to the motion evaluation result in step S112. The recommended range may be based on values common to all users, or based on values obtained from the past results of the user U and stored in the user information (434 in FIG. 7) of the user U.

When there is a conversation, the conversation in (B) is an example of a conversation in which the system responds to the utterances of a person, as indicated by “User” and “Auto”. This utterance corresponds to the case where speech information in the text format is obtained in step S106 or S108 in FIG. 6 and generated based on the information, in step S115. Such utterances may be generated by branching logic, but are preferably generated by the machine learning model 260 such as the LLM.

The generated utterance information is converted into a speech signal by the speech synthesizer 208 and output from the speaker 5. Alternatively, instead of the speech output or together with the speech output, the on-screen text creator 206 outputs an image as an on-screen text to the display 4. An image such as an illustration may be displayed in accordance with the on-screen text.

The exercise support system 100 according to the embodiment of the present disclosure has been described above. In the above configuration, utterance information related to the exercise support for the user can be output according to reaction information and motion information from the user. Thus, it is possible to provide an information processing apparatus, information processing system, method, and program for supporting the exercise of the user by carrying out dialogue appropriate to the exercise condition.

When an existing dialogue system is simply applied to the exercise support system 100, when communication is attempted between the system and the user, utterance corresponding to the exercise condition of the user cannot be made and sufficient exercise support cannot be provided. For example, in the existing dialogue system, it is possible to respond to the user's utterance based on the context of the conversation, but it is difficult to consider the exercise condition of the user.

In contrast to this, in the exercise support system 100 according to the embodiment of the present disclosure, the utterance information is generated based on the motion information of the user in addition to reaction information such as speech information, so the contents of the utterance can be made to correspond to the exercise condition of the user, and the exercise support can be suitably performed by carrying out the dialogue appropriate to the exercise condition. That is, in addition to the utterance contents to respond to the user's utterance, utterance information including utterance contents corresponding to the exercise condition of the user can be generated. For example, when the utterance contents of the user is “The weather is nice today, isn't it?”, utterance information can be generated by adding speech (“It's a pretty good pace.”) corresponding to the exercise condition (for example, depending on whether the walking pace is within or outside the appropriate range) to the usual answer (for example, “Yes. It's refreshing.”) to the user's utterance.

In addition to the utterance contents related to the reaction information of the user, utterance information including utterance contents corresponding to the exercise condition of the user can also be generated. For example, when the utterance content of the user is “I feel a little sluggish today” the utterance content suggests the physical state of the user, and utterance information can be generated by adding the utterance content corresponding to the exercise condition (“You are walking well enough”) to the utterance content based on the information related to the user's state (for example, “Please slow down without forcing yourself.”).

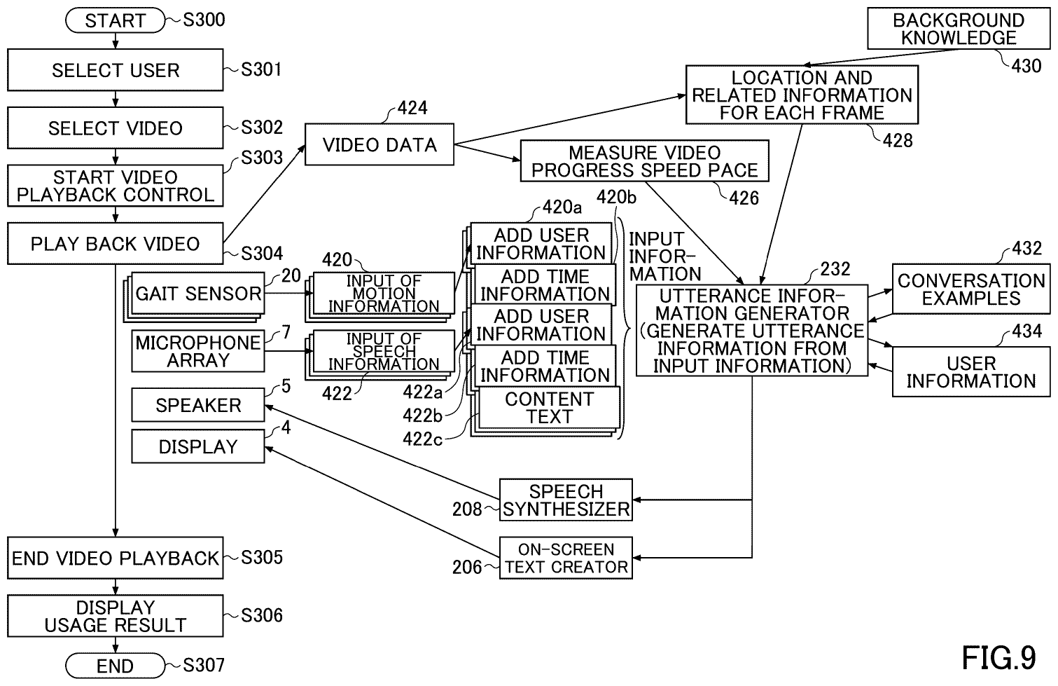

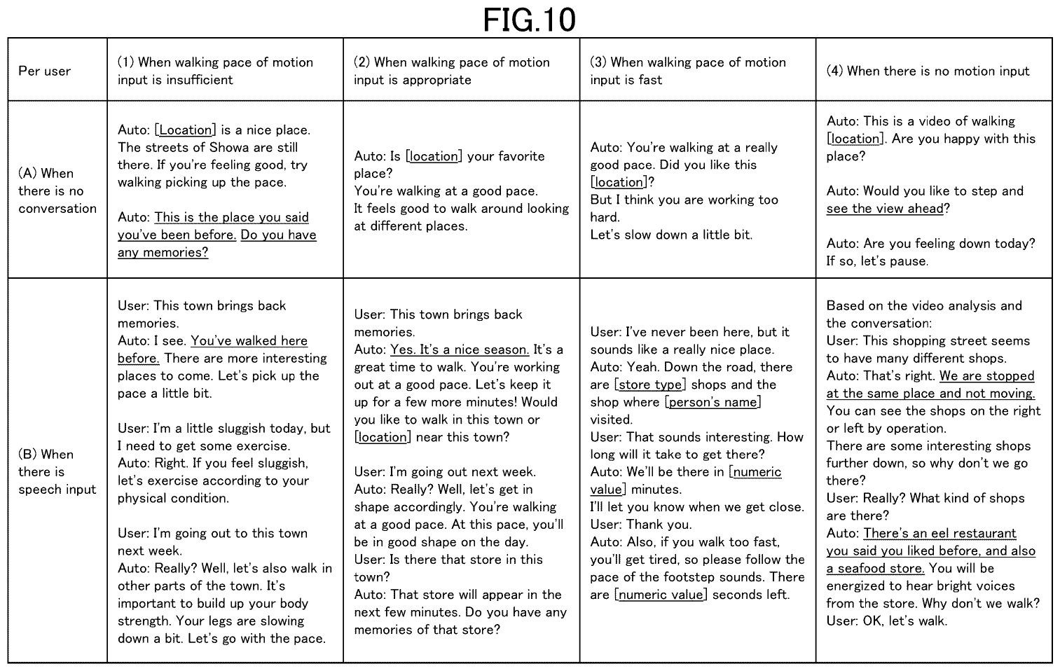

With reference to FIGS. 9 and 10, the processing of generating utterance information based on the motion information, speech information, and image attribute information will be described more specifically in the following. FIG. 9 is a diagram illustrating the utterance information generation processing based on the motion information, speech information, and image attribute information executed by the information processing apparatus 1 according to a second embodiment of the present disclosure.

FIG. 9 is a flowchart illustrating a series of flows from the start (S300) to the end (S307) of the walking exercise support processing, and data flows between blocks are illustrated next to the flowchart. As illustrated in the flowchart of FIG. 9, the walking exercise support processing starts from step S300, and in step S301, the user selection is accepted as in step S201 of FIG. 7. In step S302, the remote controller 6 accepts the selection of video content. In place of the remote controller 6, selection of the video content may be accepted by speech recognition of the video content name. In step S303, video playback control of the selected video content is started. In step S304, the video content is played back, and the user screen 310 as illustrated in FIG. 3B is displayed. In step S305, the video playback is ended. In step S306, the end screen 320 including a usage result as illustrated in FIG. 3D is displayed, and in step S307, the walking exercise support processing ends and waits for another instruction.

In the flowchart illustrated in FIG. 9, from the start of the video playback in step S304 to the end of the video playback in step S305, the motion information 420 and the speech information 422 are input as needed, as in FIG. 7. Information is added to the motion information 420 and the speech information 422.

In contrast to this, when the video is played back, video data 424 is specified, and relevant information 428 is extracted from the video data 424 by the video analyzer 242. The video analyzer 242 measures a video progress speed pace 426. The video progress speed (i.e., the walking speed at the time of shooting the video) is calculated from the analysis of the video. A frame in the video is analyzed, and image attribute information is extracted. The video progress speed is used to compare with the walking pace of the user or to control the playback speed of the video.

The utterance information generator 232 generates utterance information based on the input information and related information. At this time, the conversation examples 432 and the user information 434 may be used.

FIG. 10 is a table illustrating a conversation generated by the information processing apparatus 1 according to the second embodiment of the present disclosure based on motion information, speech information, and image attribute information. In the table illustrated in FIG. 10, as in FIG. 8, columns indicate the state of motion input and rows indicate the state of speech information input.

In the case (A) where there is no conversation, the conversation is only an utterance from the system. In the conversation example, [Location] is information obtained as image attribute information obtained from the video analysis, and a specific name such as “Asakusa” or “Mt. Takao” is included. The same applies to the [store type] and [person's name]. The [numeric value] part is obtained from, for example, the frame number associated with predetermined attribute information such as the name of the store, the progress speed of the video, the current walking pace of the user (converted from step length to speed), and the like. The underlined utterances are, for example, information generated based on the user information 434 or information generated based on the analysis of the progress speed of the video. For example, when the user information is described in the prompt statement at the beginning of the video, such a conversation is expected to be generated when the user information includes a place that has been visited by the user or a video that has been played in the past walking exercise.

The generated utterance information is similarly output by the speech synthesizer 208, the on-screen text creator 206, or both. In addition, with respect to the video, when an image reflected in the frame of the video is recognized, and when there is something that attracts the user's interest based on the user information 434, the explanatory information may be displayed as an on-screen text or an image of related information (for example, before going through a temple gate, displaying an image beyond the temple gate, etc.) may be displayed. At the end of the use, the conversation contents may be used as feedback to update the conversation examples and the user information.

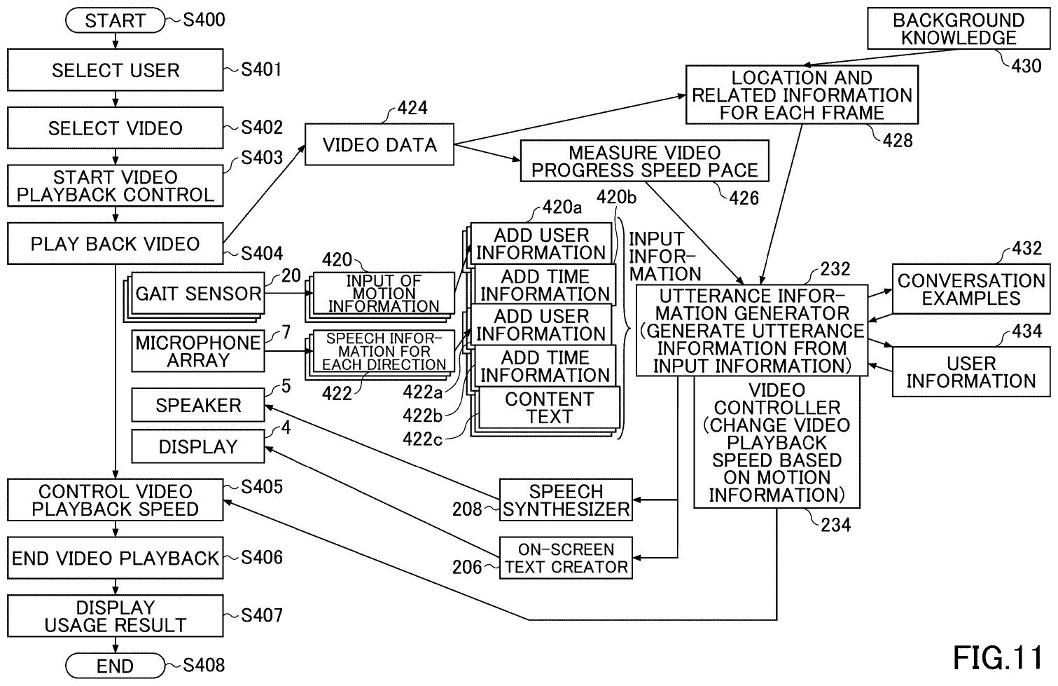

Hereinafter, with reference to FIG. 11, a video playback control processing for controlling the video playback section 226 to change the video (image information) to be output based on the motion information will be described more specifically. FIG. 11 is a diagram illustrating the video playback control based on the motion information executed by the information processing apparatus 1 according to a third embodiment of the present disclosure.

FIG. 11 is a flowchart illustrating a series of flows from the start (S400) to the end (S408) of the walking exercise support processing, and data flows between blocks are illustrated next to the flowchart. The flowchart of FIG. 11 is similar to that illustrated in FIG. 9, but the difference is that a step of controlling the video playback speed is included in step S405 from the start of the video playback in step S404 to the end of the video playback in step S406. In FIG. 11, the video controller 234 is illustrated. As in the first and second embodiments, the utterance information is generated based on the input information and related information by the utterance information generator 232, and furthermore, by the video playback section 226, the video playback speed is changed based on the motion information.

For example, as described above, the progress speed of the photographer can be estimated by analyzing the video. Then, the walking speed of the user is obtained by determining a standard step length from the walking pace of the user. Alternatively, a standard step length may be determined from the progress speed of the photographer to obtain the walking pace (hereinafter, referred to as the progress pace) of the photographer. In the following, the calculation is based on the pace for convenience. The difference (user's pace/progress pace) with the progress pace extracted from the video is calculated in accordance with the walking pace of the user during the walking exercise. When there are a plurality of users, the average of the plurality of users is calculated, and the video playback speed can be adjusted such that the average pace (speed) and the progress pace (speed) match with the average value (or a value such as the maximum value, the minimum value, etc.).

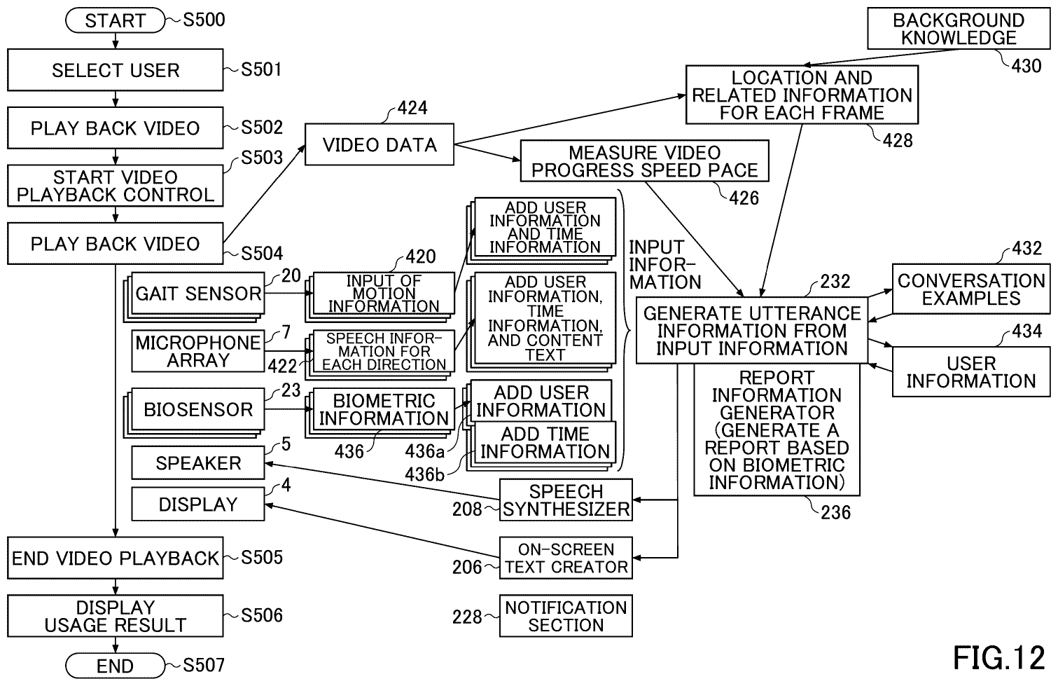

Hereinafter, with reference to FIG. 12, abnormality report processing in which the report information generator 236 generates report information based on biometric information will be described more specifically. FIG. 12 is a diagram illustrating the abnormality report processing based on the biometric information executed by the information processing apparatus 1 according to a fourth embodiment of the present disclosure.

FIG. 12 is a flowchart illustrating a series of flows from the start (S500) to the end (S507) of the walking exercise support processing, and data flows between blocks are illustrated next to the flowchart. The flowchart of FIG. 12 is similar to that illustrated in FIG. 9, and the difference is that the biosensor 23 inputs biometric information 436 as needed from the start of the video playback in step S504 to the end of the video playback in step S505. The biometric information analyzer 224 adds user information 436a and time information 436b to the biometric information 436, and transmits them to the utterance information generator 232. FIG. 12 also illustrates the report information generator 236, in which utterance information is generated by the utterance information generator 232 based on input information and related information as in the first to third embodiments. In addition, the report information generator 236 detects an abnormality based on the biometric information and creates a report.

When an abnormality is detected, such as the heart rate measured by the biosensor 23 exceeds the upper limit value of a corresponding user, the utterance information generator 232 generates speech information to advise the user to discontinue the use, and the report information generator 236 generates emergency report information, calls the notification section 228, and transmits the emergency report information to a predetermined destination. In addition, the progress of the device is stopped as necessary. The upper limit value as a reference for such an emergency report is given as user information for each user as information based on advice from a doctor, for example.

With the above configuration, the physical condition change of the user can be detected by using real-time biometric information to promote exercise more effortlessly. In addition, instead of the above biometric information, when it is detected, based on the speech information, that a predefined word or phrase (such as a word for requesting an SOS) in a predetermined list is included in the speech information in the text format from the speech recognizer 212, the user may be advised to stop the use and a report may be transmitted in the same manner as described above.

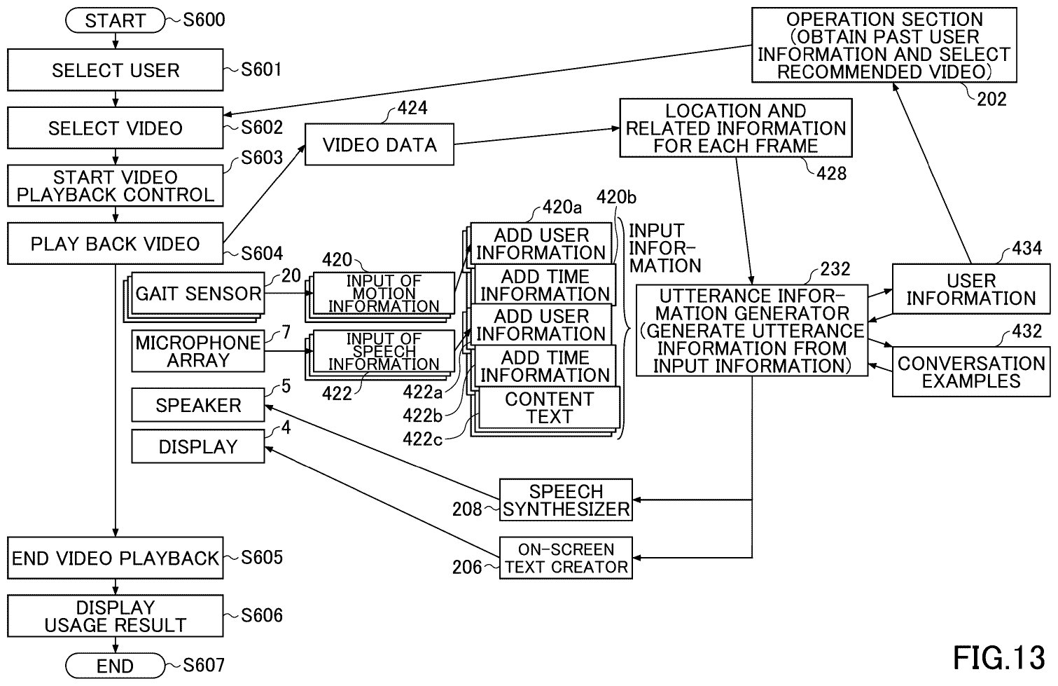

Hereinafter, with reference to FIG. 13, video content proposal processing for proposing a plurality of video contents based on past information stored in the user identification section 220 will be described more specifically. FIG. 13 is a diagram illustrating the video content proposal processing executed by the information processing apparatus 1 according to a fifth embodiment of the present disclosure.

FIG. 13 is a flowchart illustrating a series of flows from the start (S600) to the end (S607) of the walking exercise support processing. The flowchart of FIG. 13 is similar to that illustrated in FIG. 9. The difference is that the operation section 202 is illustrated as a selector in the present embodiment, and the video selection in step S602 is based on the suggestion of the recommended video content by the operation section 202. The user information 434 stores the video used in the past session, the number of steps in the session, the average walking pace, and information extracted from conversations in the past session (hobbies, experiences, friends, acquaintances, hometown, occupation, sports, other experiences, interests, etc.).

Based on the information in the user information 434, the operation section 202 selects as the proposed video content the image attribute information attached to the video content and the user information having a close similarity. When multiple users participate, the similarity with the user information of the multiple users is calculated, the similarity as a whole is calculated, and the one having a high similarity is selected. The proposed content is preferably selected from a plurality of viewpoints. For example, when there are multiple users, the preference of each user is analyzed to select the video, and a number of videos suitable for the common preference of the multiple users and a number of videos suitable for the individual preference can be selected. In addition, as a display method of the videos, scores may be calculated and the videos can be arranged and displayed in the order of the scores. In this case, the proposed videos selected from the viewpoint of the overall taste of a plurality of users are displayed at the top, and the proposed videos selected from the taste of each user can be subsequently displayed, and the most recently played videos and the like can be also displayed. The user may manually decide which of the plurality of proposed contents is to be played back. Alternatively, when the user instructs an automatic selection of the video to be played back, the video ranked at the top may be selected, or the video may be selected based on rating that corresponds to the ranking.

In this way, the user can find the content that matches with the taste of the user in a short time by: recording in the user information, the status of engagement in the past exercise and conversation that has been carried out while the content of the video used in the past is played back; selecting the content that matches with the taste of the user from the plurality of video contents; and proposing the selected contents in a list.

Utterance generation processing will be described in the following with reference to a specific conversation example.

In one or multiple embodiments, the utterance information generator 232 can generate utterance information based on the user information. The processing of generating utterance information based on the user information will be described in the following with specific examples.

The utterance information generator 232 can generate utterance information by using the user information stored in the user information storing section 246 as input. As an example of conversation, the utterance information “Mr. xxx, you love playing yyy. It's a strenuous exercise like badminton.” can be generated from information such as sports experience in the user information 434. This sports experience may be, for example, information that has been input in advance, or may have been mentioned in a conversation in a past walking exercise session. By storing the user's prior information, information that the user has uttered during use, or motion information, utterance can be made according to the user's situation by using the information, and the user's conversation can be elicited.

Furthermore, in one or multiple embodiments, the utterance information generator 232 can generate utterance information leading the user to reduce the amount of exercise or stop the exercise based on at least one of the speech information or the motion information. As another example of conversation, the utterance information “Let's call it a day. You walked a lot yesterday, so I think you are getting enough exercise effect.” may be generated in response to the detection that the walking pace indicated by the motion information has exceeded the upper limit value of the recommended range, and that the speech information includes a predefined word or phrase (e.g., “I am getting tired.”). At this time, when there is only one participant, the session of this walking exercise may be terminated. When there are multiple participants, the status of the corresponding user may be changed to “rest”.

By using speech information and motion information, an utterance can be generated based on the real time physical condition and situation of the user. Accordingly, it is possible to suppress or stop the exercise performed by the user, and thus excessive workout of the user can be prevented.

In one or multiple embodiments, the utterance information generator 232 can further generate utterance information that prompts the user to change the topic based on at least one of speech information or motion information. From the motion information, the utterance information generator 232 can generate utterance information for changing the topic according to the situation such as that the walking pace exceeds the upper limit value of the recommended range, becomes less than the lower limit value, or maintains to be within the recommended range for a long time. The utterance information generator 232 can also generate utterance information for changing the topic when an attribute of exhilaration such as “I went there!” or “That's my favorite thing.” is detected by a speech sentiment analysis or a sentiment analysis of the speech information.

The utterance information for changing the topic is generated from conversation examples, location information and related information given as user information and image attribute information, and associative information associated with the location information and related information by general knowledge. In another example of conversation, the attribute of exhilaration is detected in response to the user's utterance of “The cherry blossoms in the castle park were wonderful”, and the associative information from “castle park” can be used to prompt the user to change the topic “Is it around the Imperial Palace?”. In response to the fact that the walking pace is less than the lower limit, utterance information for changing the topic such as “It's nice that you all had a happy time. It's also nice to take a break under a cherry blossom tree. Shall we take a break?” can be generated from the motion information.

In this way, based on the speech information and the motion information, it is possible to detect whether the user is enthusiastic or unenthusiastic about the walking exercise, is bored, or has lost concentration. By changing the topic according to the detection, the concentration of the user can be increased.

In one or multiple embodiments, the user information storing section 246 stores at least one of speech information or motion information from the past walking exercise session, and the utterance information generator 232 can generate utterance information by using the stored past information as input.

The utterance information generator 232 generates utterance information from the date and time of the past session, the used video content, conversations in the session, and historical information (statistical information such as accumulated information) until now, that are stored in the user information storing section 246. In another example of conversation, utterance information of “Last time, Mr. X, Ms. Y, and Mr. Z walked at a good pace.” can be generated from past motion information (number of steps and walking pace of the past walking exercise session). Furthermore, in another example of conversation, utterance information of “Last time I visited, Mr. X talked a lot about the typhoon. Was there any particular damage?” can be generated from past speech information (past topic “typhoon”). Thus, by generating utterance information from the conversation content uttered by the user in the past and the motion information when used by the user, it is possible to have a conversation with a high degree of understanding that is close to the user. In one or a plurality of embodiments, the image attribute information includes location information, and the utterance information generator 232 can generate utterance information with the related information corresponding to the location information as input.

The video data can hold geographic coordinates such as GPS information in association with a frame, and the utterance information generator 232 can collect information (for example, store information or historical building information) from the Internet based on the geographic coordinates, and create utterance information by integrating the information, conversation information, and user information. In yet another example of conversation, utterance information “The XYZ shop on the right has recently been renovated and started selling dumplings. Mitarashi dango is popular for being delicious.” can be generated from information obtained from the Internet based on the location information associated with the frame of the video. In addition to the utterance information, image information such as a photo of a store may be added to the on-screen text.

In this way, by collecting information from the outside by using the location information of the video data and displaying the information in a conversation or an image, the user can feel the atmosphere of the scene further.

In one or multiple embodiments, the user identification section 220 identifies a specific user among a plurality of users, and the utterance information generator can generate utterance information corresponding to the user identified by the user identifier. In another conversation example, utterance information “Mr. X, Ms. Y, and Mr. Z, let's go today.” and “Mr. X, you walked a lot yesterday. Do you think you can walk a lot today?” can be generated based on the user information of a specific user. “Ms. Y, it's been a week, hasn't it? How are you doing these days? You said you had caught a cold. Has your condition recovered?” can be generated based on the user information of the specific user.

In this way, by using the usage history of each user, it is possible for each user to have a conversation about his or her physical condition and current status. By having a conversation with each user based on the individual user information, conversation information, and motion information, it is possible to give the user a sense that each user is participating and being taken care of.

While the processing of generating utterance information has been described above, the function of the report information generator 236 will be described in the following based on the report processing according to another embodiment.

As described above, the report information generator 236 compares past information or predefined reference information stored in the user information storing section 246 with the input current information, and generates report information based on the comparison result.

As the report information, emergency report information and periodic report information can be mentioned. As described with reference to FIG. 12, when an abnormality based on biometric information is detected, such as when the heart rate measured by the biosensor 23 exceeding the upper limit value given as the reference information for determining necessity of urgent reporting is detected, or when the speech information in the text format received from the speech recognizer 212 including a predefined word or phrase listed in the list given as the reference information for determining necessity of urgent reporting (such as a word for requesting SOS) is detected, the report information generator 236 generates emergency report information and calls up the notification section 228.

In addition to the above, when the walking pace indicated by the motion information or the number of conversations indicated by the speech information is less than a predetermined ratio of the average value of the past or the average value of all users stored in the user information storing section 246, the report information generator 236 can transmit information to the registered destination (for example, family members, caregivers, etc.) together with information indicating that there has been a change, such as that the exercise pace has not increased more than usual or that the conversation has not been lively. In addition, when the walking pace indicated by the motion information or the number of conversations indicated by the speech information exceeds the average value of the past or the average value of all the users stored in the user information storing section 246 by a predetermined ratio, the report information can be transmitted to the registered destination together with information indicating that the activity has been more active than usual. Such a report may be transmitted during the walking exercise session to encourage manual response by caregivers or staff, for example, the report may be transmitted after the walking exercise session as a summary of the current session, or it may be transmitted as a report to a family every predetermined period such as every month.

In this way, by comparing the past conversation and motion information with the results obtained during or after the exercise, it is possible to detect a change in the physical condition and feelings of the user, and by informing such information, the time required for the caregivers to hear from or observe the user and prepare a report can be shortened.

In the embodiments described above, the utterance information is output as a speech from the speaker 5 or as an on-screen text or an image on the display 4. Another embodiment in which speech and video are output in conjunction will be described in the following.

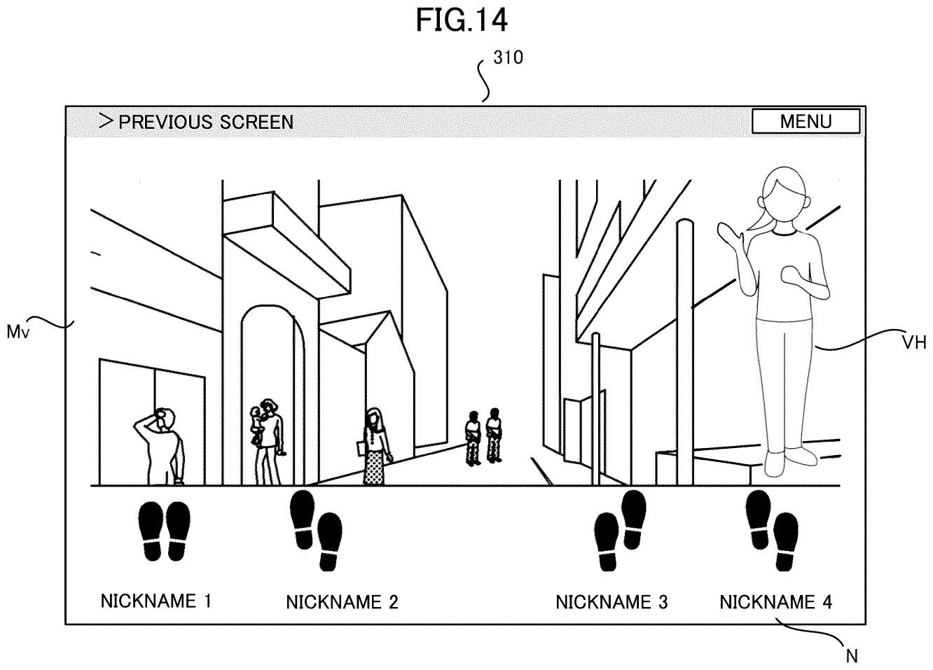

FIG. 14 is a diagram illustrating a user screen displayed on the display of the exercise support system according to another embodiment of the present disclosure. FIG. 14 is different from FIG. 3B or FIG. 3C in that a virtual human VH is displayed. Here, the virtual human is a person created by three-dimensional computer graphics. By performing speech output of utterance information while lip-syncing by using the virtual human VH, it is possible to have a conversation with a more realistic feeling. In addition, it is also possible for the virtual human VH to perform expression such as by gesture or to blink in accordance with the utterance. In this case, since the microphone array 7 includes direction information, it is possible to direct the face of the virtual human VH toward the direction of the user to whom the utterance is made or toward the direction in which a specific user is assumed to be located, or to direct the direction of the gaze. The display control of the virtual human VH is performed, for example, by the video controller 234.

Thus, by displaying a character such as a virtual human VH on the image, it is possible to bring the user into a more realistic conversation. In place of the virtual human, an animation or the like may be superimposed on the image of the video.

Thus, in the embodiment as illustrated in FIG. 14, the video controller 234 can change the content of the image information of the video by controlling the character information in conjunction with the utterance information output by the utterance information output section 204.

### (Distributed Implementation Example of Exercise Support System)

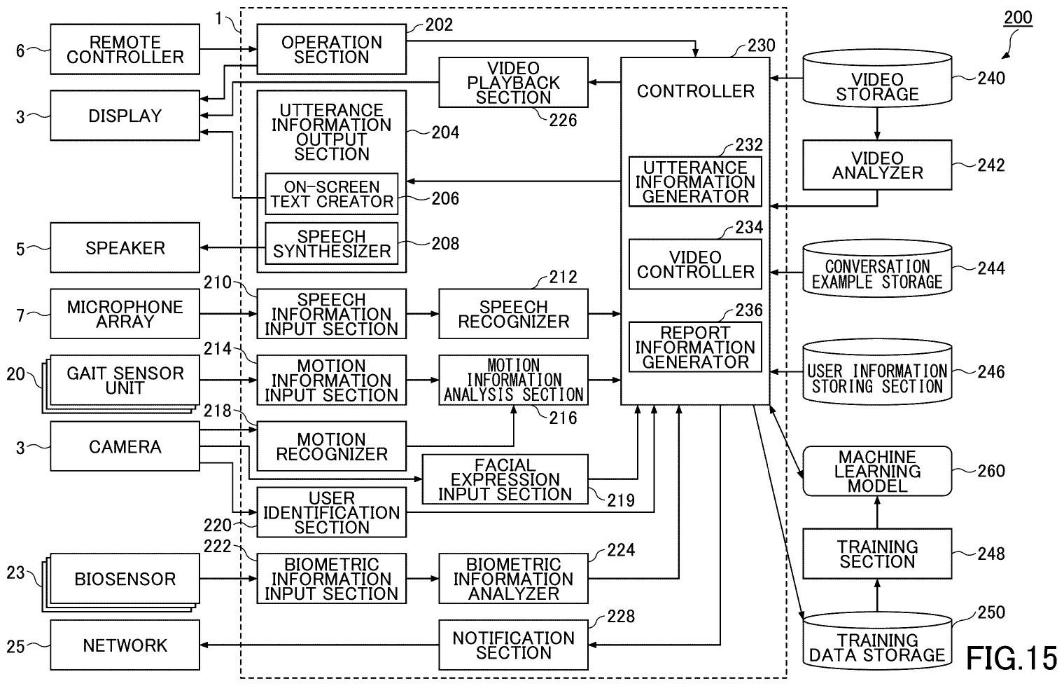

In the above description, it is assumed that among the components illustrated in FIG. 2, the components included inside the dashed rectangle 1 are implemented in the information processing apparatus 1. However, the implementation method of the exercise support system 100 is not particularly limited as described above, and various distributed implementation methods may be adopted. An example of distributed implementation of the exercise support system according to the embodiment of the present disclosure will be described in the following with reference to FIG. 15.

In the embodiment as illustrated in FIG. 15, the information processing apparatus 1 includes an operation section 202, an utterance information output section 204, a speech information input section 210, a speech recognizer 212, a motion information input section 214, a motion information analysis section 216, a motion recognizer 218, a facial expression input section 219, a user identification section 220, a biometric information input section 222, a biometric information analyzer 224, a video playback section 226, a notification section 228, and a controller 230. In contrast to this, the video storage 240, the video analyzer 242, the conversation example storage 244, the user information storing section 246, the training section 248, the training data storage 250, and the machine learning model 260 are mounted outside the information processing apparatus 1.

Distributed implementation in the form as shown in FIG. 15 can reduce the resource requirements of the information processing apparatus 1. The video storage 240, the video analyzer 242, the conversation example storage 244, the user information storing section 246, the training section 248, the training data storage 250, and the machine learning model 260 may be mounted in the same server, some or all of them may be provided in different servers, or they may be implemented by multiple servers in which one storage unit or functional unit is distributed.

The above description has been made with reference to a specific configuration of the exercise support system 100 based on the configuration as illustrated in FIG. 1. Hereinafter, a modified example of the exercise support system 100 will be described.

### (First Modified Example of Exercise Support System)

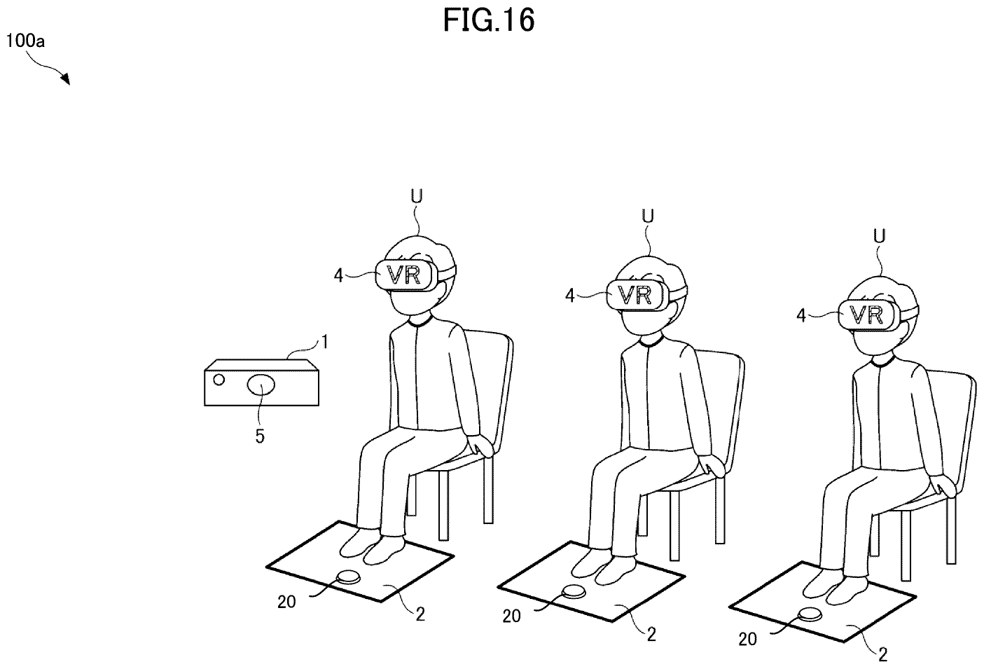

FIG. 16 is a schematic diagram illustrating an exercise support system 100a according to a first modified example. In the first modified example, the display 4 is virtual reality (VR) glasses, which is different from the embodiment in which the display 4 is a flat device. The VR glasses are an example of a worn-on-head display device. By using the VR glasses for the display 4, it is possible to give the user U a more realistic simulated experience and promote the exercise of the user U. The VR glasses may be a worn-on-head display. In addition, the VR glasses may be not limited to the goggle type as illustrated in FIG. 16, but may also be an eyeglasses type. Moreover, the image displayed on the display 4 may be a normal planar image or a 360-degrees image. For example, when the VR glasses are used for the display 4, an image obtained by cutting out a part of the 360-degrees image may be displayed in accordance with the orientation of the VR glasses.

In addition to the above, the VR glasses may be equipped with a camera (gaze-direction tracking camera) that detects the direction of the gaze from the inclination and movement of the user's eyes, and in this case, the video playback section 226 may have a function of switching the image according to the direction of the gaze. Furthermore, the gaze-direction information may be input to generate utterances. Thus, the utterance information can be changed according to the gaze-direction information of the user. For example, when it is estimated from the gaze-direction information and image information that the user is paying attention to a part of a planted tree displayed in a video of walking along a tree-lined street, an utterance related to the trees can be generated. In this case, the functions of the information processing apparatus 1 illustrated in FIG. 1 may be mounted on the VR glasses.

### (Second Modified Example of Exercise Support System)

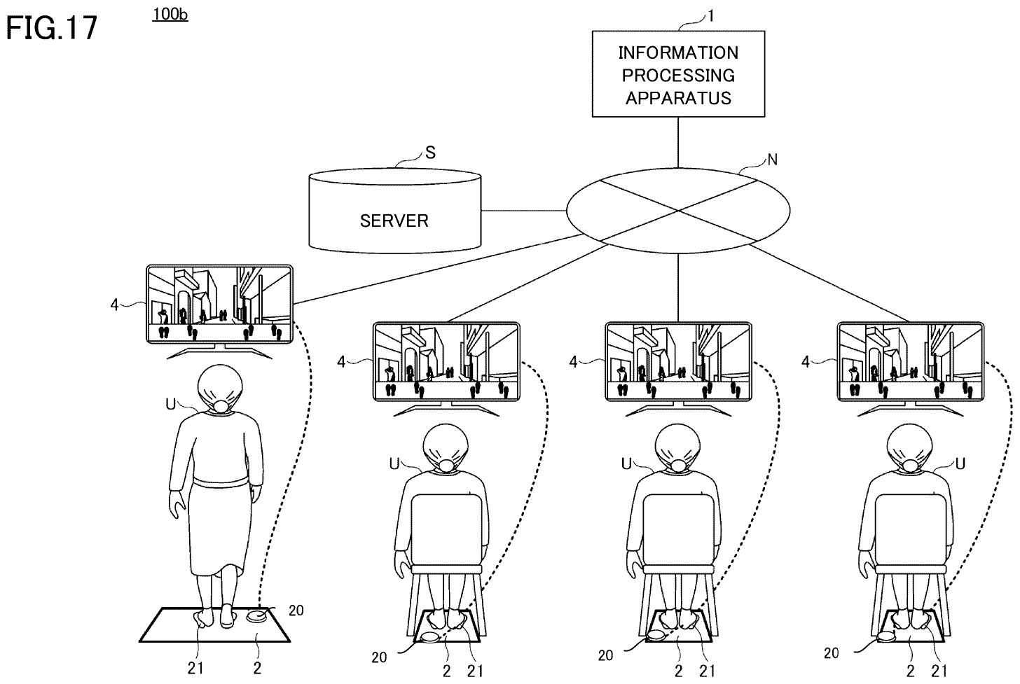

Next, an information processing apparatus or an information processing system according to a second modified example will be described. FIG. 17 is a diagram illustrating a configurational example of an exercise support system 100b according to a second modified example. The exercise support system 100b differs from the above-described embodiments and modified example in that a plurality of users U located remotely from each other can walk while sharing the same video.

In the example as illustrated in FIG. 17, the information processing apparatus 1 and the displays 4 used by the plurality of users U are connected through a network to be able to communicate with each other. Each of the displays 4 is a PC, a tablet, or a smartphone. The information processing apparatus 1 can obtain the motion information of the user U through the network and distribute the video data and the data to be displayed on the display 4 to each of the plurality of displays 4 in a streaming format.

The exercise support system 100b can provide users U, who are located remotely from each other, with a sense of realism of exercising at the same place. In the example as illustrated in FIG. 17, the gait sensor 20 is wirelessly connected to the display 4 and can transmit motion information to the information processing apparatus 1 via the display 4. However, the gait sensor 20 can also be directly connected to the network and transmit exercise state information to the information processing apparatus 1 without going through the display 4.

The data capacity of the video distributed by the exercise support system 100b may be appropriately changed depending on the devices constituting the display 4. For example, the information processing apparatus 1 can obtain information on the type of device of the display 4 used by the user U and the performance of the CPU and memory from the display 4 and distribute a video with the data capacity suitable for each device. The data capacity can be adjusted by adjusting the resolution, a frame rate, and a bit rate. Thus, even when the plurality of users U use devices of various processing speeds, since the video is distributed after reducing its data capacity for the display 4 whose processing speed is not fast, all the users can participate in the exercise while watching the common video.

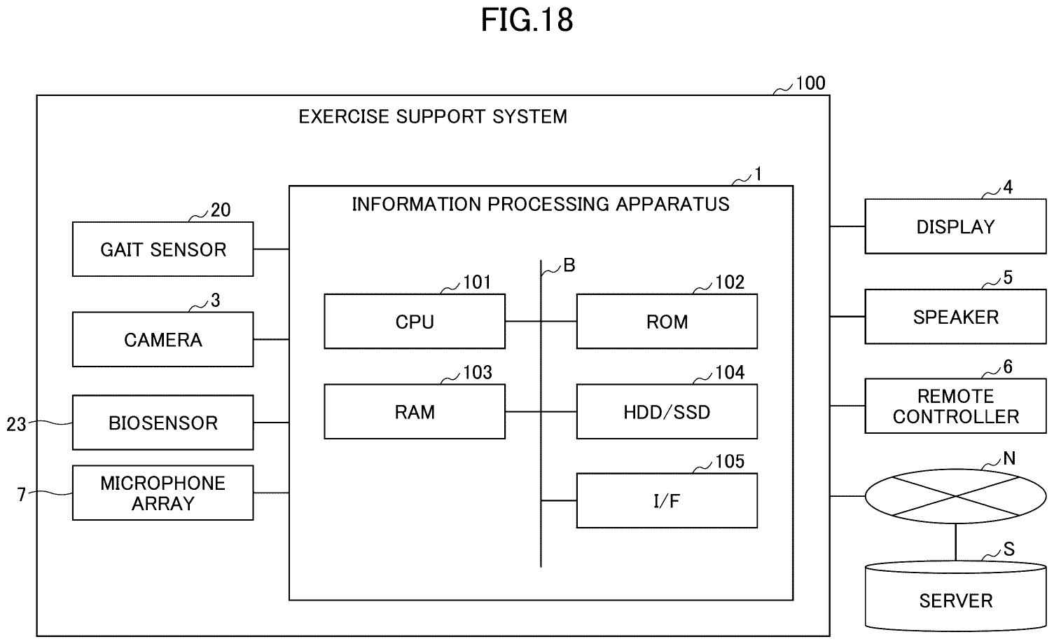

FIG. 18 is a block diagram illustrating an example of the hardware configuration of the information processing apparatus 1. The information processing apparatus 1 includes, for example, a computer. The information processing apparatus 1 includes a central processing unit (CPU) 101, a read only memory (ROM) 102, a random access memory (RAM) 103, a hard disk drive (HDD)/solid state drive (SSD) 104, and an interface (I/F) 105. These devices are intercommunicably connected via a system bus B.

The CPU 101 executes control processing including various types of arithmetic processing. The ROM 102 is a nonvolatile memory that stores programs used to drive the CPU 101 such as an initial program loader (IPL). The RAM 103 is a volatile memory used as a work area of the CPU 101. The HDD/SSD 104 is a nonvolatile memory that can store various types of information and programs used for control by the information processing apparatus 1.

The I/F 105 is an interface for communicating between the information processing apparatus 1 and equipment or devices other than the information processing apparatus 1. The I/F 105 can also communicate with external devices other than the information processing apparatus 1 via a network or the like. The external devices include the gait sensor 20, the camera 3, the display 4, the speaker 5, the remote controller 6, the microphone array 7, a lighting device (not illustrated), an air conditioner, an odor generator, and an air blower, and can output control signals to each of them. The external device may be a server S or the like communicably connected via a network.

The functions provided by the information processing apparatus 1 can also be achieved by one or a plurality of processing circuits. Here, the “processing circuit” includes devices such as an application specific integrated circuit (ASIC), a field-programmable gate array (FPGA), a digital signal processor (DSP), and conventional circuit modules designed to execute the functions described above. A part of the functions provided by the information processing apparatus 1 can also be achieved by an external device such as an external personal computer (PC) communicably connected to the information processing apparatus 1, the server S, or the like. Furthermore, a part of the functions provided by the information processing apparatus 1 can also be achieved by distributed processing between the information processing apparatus 1 and these external devices.

The embodiment of the present disclosure includes a program. The program causes a computer to perform the processing described above. With such a program, the same effects as those of the information processing apparatus 1 and the exercise support system 100 described above can be obtained.

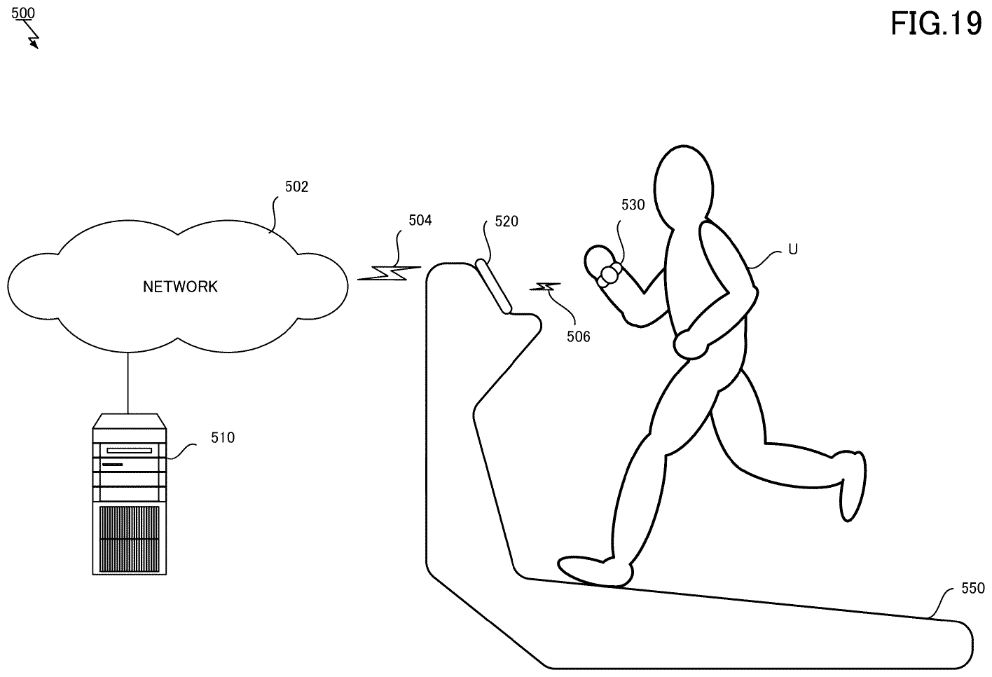

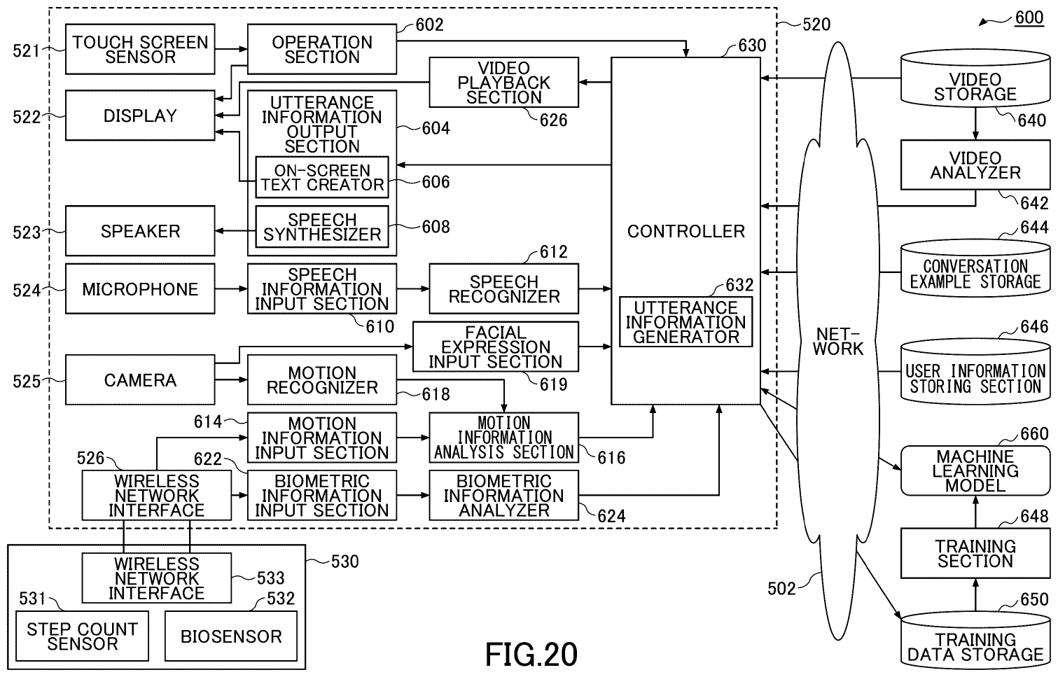

Referring to FIGS. 1 to 18, the exercise support system 100 for supporting walking exercise in a facility for the elderly has been described above. However, the exercise support system according to the embodiment of the present disclosure is not limited to the one for supporting walking exercise. Referring to FIGS. 19 and 20, an exercise support system 500 according to another embodiment supporting other kinds of physical exercise will be described in the following.

FIG. 19 is a schematic diagram illustrating the exercise support system 500 according to another embodiment of the present disclosure. The exercise support system 500 supports indoor running exercise of the user U using a treadmill 550. In the embodiment to be described, the case where “indoor running exercise” is performed as a physical exercise will be described as an example, and the “indoor running exercise” includes a pseudo running exercise in which the user's feet are raised and lowered at a predetermined position in accordance with the rotation of the running surface of the treadmill 550.

As illustrated in FIG. 19, the exercise support system 500 includes a display terminal 520 such as a smartphone or a tablet PC owned by a user and a wearable terminal 530 such as a smartwatch. The display terminal 520 includes a display as described in the following, and is installed at a position on the treadmill 550 where, for example, the user U can readily view the display. In the embodiment to be described, the display terminal 520 is installed on the treadmill 550 as a separate member from the treadmill 550. This assumes a use case in which the user installs the user's own display terminal 520 on the treadmill 550. However, the embodiment is not limited to this, and the display terminal 520 may be a device including a display provided on the treadmill 550. The user U wears the wearable terminal 530 and performs an indoor running exercise by using the treadmill 550 while watching a video (for example, a video of an instructor's exemplary movements) displayed on the display of the display terminal 520. Connection is established (paired) between the display terminal 520 and the wearable terminal 530 by a wireless connection 506 such as Wi-Fi (registered trademark) or Bluetooth (registered trademark).

During the indoor running exercise of the user U, the wearable terminal 530, as an example of a motion information obtaining device, obtains motion information (information on running and walking exercises) related to the exercise, and, as an example of the reaction information obtaining device, obtains the biometric information (heart rate, blood oxygen level, and activity level) of the user, and outputs them to the display terminal 520 via the wireless connection 506. During the indoor running exercise of the user U, the wearable terminal 530, as an example of the reaction information obtaining device, may also obtain speech information of the utterance of the user U and output it to the display terminal 520 via the wireless connection 506.

The display terminal 520 is further connected to a network 502 such as the Internet via a mobile communication network 504 such as 4G or 5G. A server device 510 is arranged in the network 502. The display terminal 520 communicates with the server device 510 via the mobile communication network 504 and the network 502 to provide the exercise support function described above to the user U.

As will be described in the following, the display terminal 520 also includes a speaker, outputs speech based on the generated utterance information, and approaches the user U. In addition, the display terminal 520, as an example of the reaction information obtaining device, may obtain speech information of the utterance of the user U by using a microphone provided by the display terminal 520 during the indoor running exercise of the user U. The display terminal 520 may also be provided with a camera guided to take a picture of the use environment as well as the user U while they are included in the field of view of the camera, and may analyze an image input by the camera provided by the display terminal 520, as an example of the motion information obtaining device or reaction information obtaining device, during the running exercise of the user U to detect facial expression of the user U and obtain facial expression information, or to detect the skeletal structure of the user U and perform motion analysis to obtain motion information.

In the exercise support system 500 as illustrated in FIG. 19, the user U performs an indoor running exercise by using the treadmill 550 while watching a video. During the indoor running exercise by the user U, the exercise support system 500 generates utterance information based on motion information and reaction information (speech information, biometric information, or facial expression information obtained from the user U within a predetermined time period, in response to the speech output of the generated speech information) of the user U, and encourages the user. In this way, exercise support suitable for the exercise condition of the user is performed.

In the embodiment to be described, it is assumed that one user U uses one display terminal 520 and one wearable terminal 530, but the present embodiment is not limited. In other embodiments, a plurality of users U may be configured to utilize the exercise support system 500.

In the example as illustrated in FIG. 19, indoor running is described as an example of physical exercise, but the type of physical exercise supported by the exercise support system 500 according to the embodiment of the present disclosure is not particularly limited. As the physical exercise, in addition to the above-described running exercise such as indoor running, various exercises performed for the purpose of maintaining and enhancing health and physical strength such as weight training, aerobics, dance, stretching, yoga, and fitness may be mentioned.

The functional configuration of the exercise support system 500 will be described more specifically in the following with reference to FIG. 20. FIG. 20 is a diagram illustrating functional blocks 600 of the exercise support system 500 according to another embodiment of the present disclosure.

As hardware included in the display terminal 520, FIG. 20 includes a touch screen sensor 521, a display 522, a speaker 523, a microphone 524, a camera 525, and a wireless network interface 526. The functional blocks 600 of the display terminal 520 as illustrated in FIG. 20 include an operation section 602, an utterance information output section 604, a speech information input section 610, a speech recognizer 612, a motion information input section 614, a motion information analyzer 616, a motion recognizer 618, a facial expression input section 619, a biometric information input section 622, a biometric information analyzer 624, a video playback section 626, and a controller 630.

In FIG. 20, the hardware included in the wearable terminal 530 is also illustrated. The wearable terminal 530 includes a step count sensor 531, a biosensor 532, and a wireless network interface 533.

FIG. 20 also includes a video storage 640, a video analyzer 642, a conversation example storage 644, a user information storing section 646, a training section 648, a training data storage 650, and a machine learning model 660 as external functional blocks of the display terminal 520, such as functional blocks on the server device 510 to which the display terminal 520 is connected via the network 502.

The functional sections described with reference to FIG. 20 are the same as those illustrated in FIG. 2.

The controller 630 performs overall processing and control for providing exercise support for the user U, including processing for generating utterance information. The controller 630 includes an utterance information generator 632 as in the embodiment as illustrated in FIG. 2. The utterance information generator 632 generates utterance information based on input information that is input through input sections such as the speech information input section 610, the motion information input section 614, the facial expression input section 619, and the biometric information input section 622, and outputs the utterance information to the utterance information output section 604. The controller 630 can also control video playback by the video playback section 626 based on the input information. The controller 630 can also store in the user information storing section 646, which will be described in the following, the contents of conversation performed during the running exercise, the state of engagement in the walking exercise, and topic information extracted from the conversation contents in accordance with the running exercise performed by the user U.

The speech information input section 610 receives the input of speech information from the usage environment via the microphone 524 and outputs the speech information to the speech recognizer 612. The speech recognizer 612 applies the STT conversion to speech information in the digital speech signal format from the speech information input section 610, converts it into speech information in the text format, and outputs it to the controller 230. As described above, the speech recognizer 212 may perform speech emotion recognition, natural language analysis, and the like in addition to the above. In the embodiment to be described, it is assumed that the display terminal 520 includes the microphone 524, and the speech information input section 610 receives input of speech information from the microphone 524. However, it is not limited to the present embodiment, and the speech information may be obtained by the microphone provided with the wearable terminal 530. In this case, the speech information received from the wearable terminal 530 is input to the speech information input section 610 via the wireless network interfaces 526 and 533.

The motion information input section 614 receives input of motion information related to the running exercise of the user U from the step count sensor 531 included in the wearable terminal 530 via the wireless network interface 526, and inputs the input to the motion information analysis section 216. The motion information is the same, and thus description thereof is omitted. Also, as described above, the method of obtaining the motion information of the user is not particularly limited. For example, a device including an acceleration sensor or a gyro sensor included in the wearable terminal 530 can be used to analyze the gait from the pattern of acceleration and angular velocity to obtain motion information. In this case, the motion information input section 614 receives the motion information that is input via the wireless network interfaces 526 and 533. Furthermore, the motion information can be obtained by using the camera 525 provided with the display terminal 520. In this case, the motion recognizer 618 analyzes the image input from the camera 525, detects the skeletal structure of the user U, performs motion analysis to detect the walking exercise, and the motion information input section 614 receives the motion information input.

The facial expression input section 619 detects the face area of the person from the image captured by the camera 525, recognizes the facial expression of the person from the face image, and transmits the facial expression information to the controller 630.

The biometric information input section 622 receives the biometric information (wearer's heart rate, blood oxygen level, activity level, etc.) related to the body of the user U from the biosensor 532 included in the wearable terminal 530 via the wireless network interfaces 526 and 533. The biometric information analyzer 624 analyzes the biometric information, adds a timestamp or the like, and transmits the biometric information to the controller 630. As for the biosensor 532, various types of biosensors 532 can be exemplified, such as an activity meter (activity tracker), a sleep meter (sleep tracker), a blood pressure meter, and a small brain-activity sensor. A method of obtaining the biometric information is not limited to the above. As described above, there is known a technique for estimating the heart rate or respiration rate by photographing a face with a camera, and the heart rate or respiration rate estimated from the image of the face area of a person detected from the image captured by the camera 525 may be input to the biometric information input section 622.

Since the video storage 640, the video analyzer 642, the conversation example storage 644, the user information storing section 646, the controller 630 (the utterance information generator and the video controller), the training section 648, the training data storage 650, and the machine learning model 660 have the same configuration as those in the embodiment described with reference to FIG. 2, a detailed description thereof will be omitted. The video storage 640, the video analyzer 642, the conversation example storage 644, the user information storing section 646, the training section 648, the training data storage 650, and the machine learning model 660 may be provided in the same server, or some or all of them may be provided in different servers, or may be implemented by multiple servers in which one storage or functional unit is distributed. In addition, the training section 648, the training data storage 650, and the machine learning model 660 may be configured outside the exercise support system 500 and configured to communicate with the server device 510 through an API.

According to the exercise support system 500 described with reference to FIGS. 19 and 20, the user U can perform physical exercise such as indoor running exercise by using the treadmill 550 while watching a video, and can support the exercise of the user by performing dialogue suitable for the exercise condition of the user U during this physical exercise.

In the exercise support system 500 described with reference to FIGS. 19 and 20, typically, the biosensor 532 included in the wearable terminal 530 is included in a first sensor configured to obtain the reaction information of the user. When the biosensor 532 provided in the wearable terminal 530 is included in the first sensor, the wireless network interface 533 is included in a transmitter configured to transmit the obtained reaction information to the display terminal 520. In addition to the biosensor 532 included in the wearable terminal 530, at least one of the microphone 524 provided with the display terminal 520, the camera 525 provided with the display terminal 520, or a separate microphone provided with the wearable terminal 530 may be included in the first sensor configured to obtain the reaction information of the user, together with the biosensor 532 or in place of the biosensor 532.

In the embodiment described with reference to FIGS. 19 and 20, at least one of the step count sensor 531 included in the wearable terminal 530 or the camera 525 provided with the display terminal 520 may be included in a second sensor configured to obtain the motion information. When the step count sensor 531 provided with the wearable terminal 530 is included in the second sensor, the wireless network interface 533 is included in a transmitter configured to transmit the obtained motion information to the display terminal 520.

In a specific embodiment, during the indoor running exercise of the user, motion information such as the number of steps and the running distance is detected from the step count sensor 531 included in the wearable terminal 530 worn by the user, and biometric information (which is reaction information) related to the user's body is detected from the biosensor 532 included in the wearable terminal 530. Then, the wireless network interface 533 of the wearable terminal 530 transmits the motion information and the biometric information to the display terminal 520. The motion information from the step count sensor 531 is input to the motion information input section 614 of the display terminal 520, and the biometric information from the biosensor 532 is input to the biometric information input section 622. As described with reference to FIG. 2, utterances are generated by the utterance information generator 632 included in the controller 630, and the generated utterance information is output from the utterance information output section 604 through the speaker 523 and the display 522. As a result, it is possible to engage in dialogue corresponding to the exercise conditions of the user as described with reference to FIG. 8.

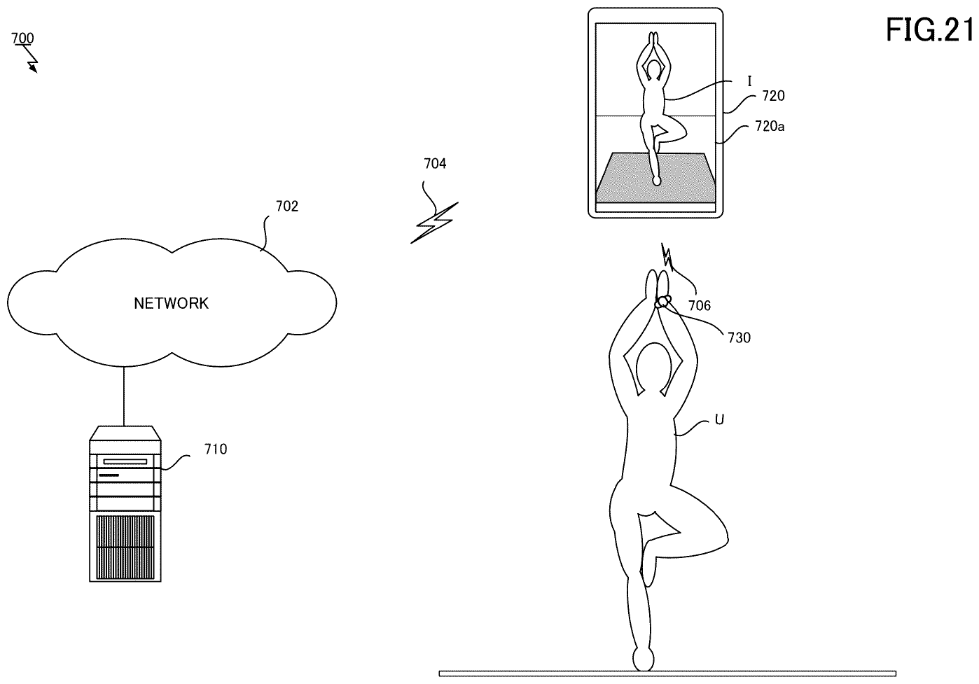

In the embodiment as described with reference to FIGS. 19 and 20, indoor running is described as an example of physical exercise. However, as described above, the types of physical exercise that can be supported in the embodiment of the present disclosure are not particularly limited and can be applied to fitness exercises such as stretching exercises and yoga exercises that do not involve walking, running, and stepping exercises. Hereinafter, with reference to FIG. 21, an exercise support system 700 according to another embodiment that supports yoga exercises (similarly in stretching exercises) that do not involve walking, running, and stepping exercises as another type of physical exercise will be described.

FIG. 21 is a schematic diagram illustrating an exercise support system 700 according to another embodiment of the present disclosure. The exercise support system 700 supports yoga exercises of the user U. In the embodiment to be described, a case where “yoga exercises” are performed as the physical exercise will be described as an example, where “yoga exercises” typically include gymnastic exercises that are performed on a floor surface by using the whole body and do not involve walking, running and stepping exercises.

As illustrated in FIG. 21, the exercise support system 700 includes a display terminal 720 such as a smartphone or tablet PC owned by a user and a wearable terminal 730 such as a smartwatch. The display terminal 720 is provided with a display 720a as described in the following, and the user U sets the display 720a at a position where the user can readily view it. The user U wears the wearable terminal 730 and performs yoga exercises while watching a video (for example, a video of an exemplary movement shown by an instructor I) displayed on the display of the display terminal 720. Connection (pairing) is established between the display terminal 720 and the wearable terminal 730 by a wireless connection 706 such as Wi-Fi (registered trademark) or Bluetooth (registered trademark).

During the yoga exercise of the user U, the wearable terminal 730 obtains motion information (for example, information of the arm motion that is linked to the motion of the arm, measured by a provided momentum sensor such as an acceleration sensor or a gyro sensor) related to the exercise and biometric information (heart rate, blood oxygen level, and activity level) of the user, and outputs them to the display terminal 720 via the wireless connection 706. The wearable terminal 730 may also obtain speech information of the utterance of the user U during the yoga exercise of the user U, and output the speech information to the display terminal 720 via the wireless connection 706.

The display terminal 720 is further connected to a network 702 such as the Internet via a mobile communication network 704 such as 4G or 5G. A server device 710 is arranged on the network 702. The display terminal 720 provides the above-described exercise support function to the user U by communicating with the server device 710 via the mobile communication network 704 and the network 702.

The display terminal 720 may also include a speaker, and outputs sound based on the generated utterance information to approach the user U. In addition, the display terminal 720 may obtain speech information of the utterance of the user U by the microphone provided with the display terminal 720 during the yoga exercise of the user U. The display terminal 720 may also include a camera guided to take a picture including the use environment and the user U by including them in the field of view of the camera. The display terminal 720 may obtain facial expression information by analyzing the image input by the camera provided with the display terminal 720, or may obtain motion information related to the motion of the user U by detecting the skeletal structure of the user U and performing motion analysis, during the running exercise of the user U.

In the exercise support system 700 as illustrated in FIG. 21, the user U performs a yoga exercise while viewing a video. During the yoga exercise by the user U, the exercise support system 700 generates utterance information based on the motion information and reaction information (speech information, biometric information, or facial expression information from the user U within a predetermined time in response to the speech output of the generated utterance information) of the user U and encourages the user with the information. Thus, the exercise support suitable for the exercise condition of the user is performed.

In the embodiment to be described, it is assumed that one user U uses one display terminal 720 and one wearable terminal 730, but the embodiment is not limited thereto. In other embodiments, the exercise support system 700 may be configured such that a plurality of users U can use the exercise support system 700. In the embodiment described with reference to FIG. 21, the functional blocks are the same as those described with reference to FIG. 20 except that the symbols are different, and the description thereof is omitted.

In the exercise support system 700 described with reference to FIG. 21, the biosensor included in the wearable terminal 730 constitutes the first sensor for obtaining the reaction information of the user. When the biosensor included in the wearable terminal 730 is included in the first sensor, the wireless network interface provided with the wearable terminal 730 is included in a transmitter configured to transmit the obtained reaction information to the display terminal 720. In addition to the biosensor included in the wearable terminal 730, at least one of the microphone provided with the display terminal 720, the camera provided with the display terminal 720, or the separate microphone provided with the wearable terminal 730 may be included in the first sensor configured to obtain the reaction information of the user together with or in place of the biosensor.

In the embodiment described with reference to FIG. 21, at least one of the momentum sensor included in the wearable terminal 730 or the camera provided with the display terminal 720 may be included in the second sensor configured to obtain the motion information. When the step count sensor provided with the wearable terminal 730 is included in the second sensor, the wireless network interface provided with the wearable terminal 730 is included in the transmitter for transmitting the obtained motion information to the display terminal 720. When the momentum sensor provided with the wearable terminal 730 is included in the second sensor, the motion information is the detected acceleration or angular velocity, or motion analysis information obtained by analyzing the acceleration and angular velocity. When the camera provided with the display terminal 720 is included in the second sensor, the motion information is the information of the user body movement detected by analyzing the image captured by the camera.

In a specific embodiment, during the yoga exercise of the user, motion information such as arm movement is detected by the momentum sensor provided with the wearable terminal 730 worn by the user, or motion information is detected from the analysis of the image captured by the camera provided with the display terminal 720, and biometric information (reaction information) related to the user's body is detected by the biosensor provided with the wearable terminal 730. Then, the wireless network interface of the wearable terminal 730 transmits the motion information and the biometric information to the display terminal 720. Motion information from the momentum sensor included in the wearable terminal 730 or the camera provided with the display terminal 720 is input to the motion information input section of the display terminal 720, and biometric information from the biosensor included in the wearable terminal 730 is input to the biometric information input section, and as described with reference to FIG. 2, utterance is generated by the utterance information generator included in the controller as illustrated in FIG. 20, and the generated utterance information is output from the utterance information output section through a speaker or a display. As a result, it is possible to carry out dialogue corresponding to the exercise condition of the user as described with reference to FIG. 8.

According to the embodiment described above, it is possible to output utterance information related to the exercise support for the user in accordance with reaction information and motion information from the user. As a result, it is possible to provide an information processing apparatus or an information processing system which suitably supports the exercise of the user by carrying out dialogue suitable for the exercise support.

In addition, the exercise support system 100 was used in order for the user to carry out walking exercise while watching a video as a part of rehabilitation or recreation in the above-described facility for the elderly. In particular, by pseudo locomotion with feet, which the elderly have been doing for many years, and combining it with stimuli such as videos, it is possible to enhance the user's sense of immersion and add elements of physical movement. In turn, it is possible to implement rehabilitation and recreation for maintaining the brain health and physical health of the elderly while reducing the intervention of caregivers.

In the above-described embodiments, walking exercise is used as an example of physical exercise, and rehabilitation or recreation in facilities for the elderly is mainly described as an application. However, physical exercise is not limited to walking exercise, nor is it limited to rehabilitation or recreation in facilities for the elderly. It can be applied to various physical exercises that move the body, and applications include experiential event applications in events, exhibitions, tourist facilities, and public facilities, medical rehabilitation systems in rehabilitation facilities for maintenance and recovery after illness and nursing homes, facility tour experience tools for education, and online games.

With the above configuration, it is possible to support the exercise of the user by achieving suitable interaction with the user based on the exercise condition of the user.

Although preferred embodiments have been described in detail above, the present disclosure is not limited to the above-described embodiments, and various modifications and substitutions may be made to the above-described embodiments of the present disclosure without departing from the scope of claims.

All figures such as ordinals and quantities used in the description of the embodiments of the present disclosure are exemplified for the purpose of specifically explaining the technique of the present disclosure, and the present disclosure is not limited to the exemplified figures. Furthermore, the connection relationships between the components are exemplified for the purpose of specifically explaining the technique of the present disclosure, and the connection relationships that achieve the functions of the present disclosure are not limited thereto.

The division of blocks in the functional block diagram is an example, and a plurality of blocks may be achieved as one block, one block may be divided into a plurality of blocks, or a part of a function may be transferred to another block. In addition, a single hardware or software may process the functions of a plurality of blocks having similar functions in parallel or time division. In addition, a part or all of the functions may be distributed to a plurality of computers.

Embodiments of the present disclosure are, for example, as follows.

<1> An information processing apparatus, including:
a motion information inputter to which motion information related to a physical exercise of a user is input;
a reaction information inputter to which reaction information related to a reaction of the user, the reaction information being different from the motion information, is input; and
an utterance information outputter configured to output utterance information related to exercise support for the user based on the reaction information that is input to the reaction information inputter and the motion information that is input to the motion information inputter.

<2> The information processing apparatus according to <1>, further including:
an utterance information generator configured to generate the utterance information by using the reaction information that is input to the reaction information inputter and the motion information that is input to the motion information inputter as inputs.

<3> The information processing apparatus according to <2>, wherein
the reaction information includes at least one of speech information of utterance of the user, facial expression information indicating a facial expression of the user, or biometric information related to a body of the user.

<4> The information processing apparatus according to <2>, further including:
a video playback section configured to control playback of a video to be displayed on a display, wherein
the utterance information generator generates the utterance information by further using at least one of image information included in the video or attribute information associated with the image information as an input.

<5> The information processing apparatus according to <4>, further including:
an image controller configured to control change of playback speed of the video; change of contents of the image information of the video; or insertion of one or both of an on-screen text and an insertion image into an image of the video, based on the motion information that is input to the motion information inputter.

<6> The information processing apparatus according to any one of <2> to <5>, further including:
a user information storage configured to store user information related to the user, wherein
the utterance information generator generates the utterance information by using the user information stored in the user information storage as an input.

<7> The information processing apparatus according to any one of <2> to <6>, wherein
the utterance information generator is configured to generate the utterance information that leads to reduce an amount of exercise; the utterance information that leads to stop the exercise; or the utterance information that leads to change a topic, based on at least one of the reaction information or the motion information.

<8> The information processing apparatus according to any one of <1> to <6>, further including:
a biometric information inputter to which biometric information related to a body of the user is input; and
a notifier configured to perform notification upon detecting an abnormality based on the biometric information.

<9> The information processing apparatus according to any one of <2> to <8>, further including:
an information storage configured to store at least one of the reaction information or the motion information, wherein
the utterance information generator generates the utterance information by using information from a past stored in the information storage as an input.

<10> The information processing apparatus according to any one of <1> to <9>, further including:
an information storage configured to store at least one of the reaction information or the motion information; and
a selector configured to select a video content to be proposed based on information from a past stored in the information storage.

<11> The information processing apparatus according to any one of <1> to <10>, further including:
an information storage configured to store at least one of the reaction information or the motion information; and
a report information generator configured to generate report information based on a comparison result obtained by comparing information from a past stored in the information storage and input current information.

<12> The information processing apparatus according to <4>, wherein
the image information includes location information, and
the utterance information generator generates the utterance information by using related information corresponding to the location information as an input.

<13> The information processing apparatus according to <5>, wherein
the image information includes character information superimposed on an image, and
the image controller changes a content of the image information of the video by controlling the character information in conjunction with the utterance information output by the utterance information outputter.

<14> The information processing apparatus according to any one of <2> to <13>, further including:
a user identifier configured to identify a specific user among a plurality of users, wherein
the utterance information generator is configured to generate the utterance information that corresponds to the specific user identified by the user identifier.

<15> The information processing apparatus according to any one of <2> to <14>, wherein
the utterance information generator is configured to generate the utterance information based on a machine learning model.

<16> The information processing apparatus according to <1>, further including:
an utterance information generator configured to generate the utterance information based on branching logic by using the reaction information that is input to the reaction information inputter and the motion information that is input to the motion information inputter as inputs.

<17> The information processing apparatus according to any one of <1> to <16>, wherein
the utterance information outputter is configured to output the utterance information by speech, letters displayed in an image, sign language, or machine movement displayed in the image.

<18> The information processing apparatus according to any one of <1> to <17>, further including:
a motion information analyzer configured to generate data including at least one type of information on a time length of walking exercise, number of steps in a predetermined period, average walking speed of predetermined time points or of a predetermined period, or an intensity of the walking exercise.

<19> The information processing apparatus according to any one of <2> to <18>, further including:
a motion information analyzer configured to generate evaluation information of a physical exercise from the motion information that is input to the motion information inputter, wherein
the utterance information generator is configured to generate the utterance information by using the evaluation information as the motion information.

<20> An information processing system, including:
a motion information obtaining device configured to obtain motion information related to a physical exercise of a user;
a reaction information obtaining device configured to obtain reaction information related to a reaction of the user, the reaction information being different from the motion information; and
an information processing apparatus configured to output utterance information related to exercise support for the user based on the reaction information obtained by the reaction information obtaining device and the motion information obtained by the motion information obtaining device.

<21> An information processing system, including:
a wearable terminal to be worn by a user; and
a display terminal including a display on which a video related to a physical exercise is displayed, wherein
the wearable terminal includes a transmitter configured to transmit reaction information obtained from the user to the display terminal, and
the display terminal includes
   a motion information inputter to which motion information related to a physical exercise of the user, the motion information being different from the reaction information, is input;
   a reaction information inputter to which the reaction information received from the wearable terminal is input; and
   an utterance information outputter configured to output utterance information related to exercise support for the user based on the reaction information that is input to the reaction information inputter and the motion information that is input to the motion information inputter.

<22> The information processing system according to <21>, wherein
the wearable terminal includes
   a first sensor configured to obtain the reaction information related to a reaction of the user; and
   a second sensor configured to obtain the motion information related to the physical exercise of the user, and
the transmitter is configured to transmit the reaction information obtained by the first sensor and the motion information obtained by the second sensor to the display terminal.

<23> The information processing system according to <21>, wherein
the display terminal includes an image capturer configured to capture an image, and
the motion information is movement information related to movement of the user obtained by analyzing the image.

<24> A non-transitory computer-readable recording medium storing a program causing a computer to function as:
a motion information inputter to which motion information related to a physical exercise of a user is input,
a reaction information inputter to which reaction information related to a reaction of the user, the reaction information being different from the motion information, is input, and
an utterance information outputter configured to output utterance information related to exercise support for the user based on the reaction information that is input to the reaction information inputter and the motion information that is input to the motion information inputter.

<25> A method executed by computer processing, the method including:
inputting motion information related to a physical exercise of a user;
inputting reaction information related to a reaction of the user; and
outputting utterance information related to exercise support for the user based on the reaction information and the motion information.

The functionality of the elements disclosed herein may be implemented by using circuitry or processing circuitry which includes general purpose processors, special purpose processors, integrated circuits, application specific integrated circuits (ASICs), digital signal processors (DSPs), field programmable gate arrays (FPGAs), conventional circuitry and/or combinations thereof which are configured or programmed to perform the disclosed functionality. Processors are considered as processing circuitry or circuitry as they include transistors and other circuitry therein. In the present disclosure, the circuitry, units, or means are hardware that carry out or are programmed to perform the recited functionality. The hardware may be any types of hardware disclosed herein or otherwise known which is programmed or configured to carry out the recited functionality. When the hardware is a processor which may be considered as a type of circuitry, the circuitry, means, or units are a combination of hardware and software, the software being used to configure the hardware and/or processor.

What is claimed is:

1. An information processing apparatus, comprising:
circuitry configured to
input motion information related to a physical exercise of a user;
input reaction information related to a reaction of the user; and
output utterance information related to exercise support for the user based on the reaction information and the motion information.

2. The information processing apparatus according to claim 1, further comprising:
the circuitry configured to
generate the utterance information by using the reaction information and the motion information.

3. The information processing apparatus according to claim 2, wherein
the reaction information includes at least one of speech information of utterance of the user, facial expression information indicating a facial expression of the user, or biometric information related to a body of the user.

4. The information processing apparatus according to claim 2, further comprising:
the circuitry configured to control playback of a video to be displayed on a display; and
generate the utterance information by further using at least one of image information included in the video or attribute information associated with the image information.

5. The information processing apparatus according to claim 4, further comprising:
the circuitry configured to control, based on the motion information,
change of playback speed of the video;
change of contents of the image information of the video; or
insertion of one or both of an on-screen text and an insertion image into an image of the video.

6. The information processing apparatus according to claim 2, further comprising:
the circuitry configured to
store user information related to the user; and
generate the utterance information by using the stored user information.

7. The information processing apparatus according to claim 2, wherein
the circuitry is configured to, based on at least one of the reaction information or the motion information,
generate the utterance information that leads to reduce an amount of exercise;
stop the exercise; or
change a topic.

8. The information processing apparatus according to claim 1, further comprising:
the circuitry configured to
input biometric information related to a body of the user; and
perform notification upon detecting an abnormality based on the biometric information.

9. The information processing apparatus according to claim 2, further comprising:
the circuitry configured to
store at least one of the reaction information or the motion information; and
generate the utterance information by using at least one of the stored reaction information or the stored motion information.

10. The information processing apparatus according to claim 1, further comprising:
the circuitry configured to
store at least one of the reaction information or the motion information; and
select a video content to be proposed based on at least one of the stored reaction information or the stored motion information.

11. The information processing apparatus according to claim 1, further comprising:
the circuitry configured to
store at least one of the reaction information or the motion information; and
generate report information based on a comparison result obtained by comparing past stored information and current information.

12. The information processing apparatus according to claim 4, wherein
the image information includes location information, and
the circuitry is configured to generate the utterance information by using related information corresponding to the location information.

13. The information processing apparatus according to claim 5, wherein
the image information includes character information superimposed on the image of the video, and
the circuitry is configured to change a content of the image information of the video by controlling the character information in conjunction with the utterance information.

14. The information processing apparatus according to claim 2, further comprising:
the circuitry configured to
identify a specific user among a plurality of users; and
generate the utterance information that corresponds to the specific user.

15. The information processing apparatus according to claim 2, wherein
the circuitry is configured to generate the utterance information based on a machine learning model.

16. The information processing apparatus according to claim 1, further comprising:
the circuitry configured to generate the utterance information based on branching logic by using the reaction information and the motion information.

17. The information processing apparatus according to claim 1, wherein
the circuitry is configured to output the utterance information by at least one of speech, letters displayed in an image, sign language displayed in the image, or machine movement displayed in the image.

18. The information processing apparatus according to claim 1, further comprising:
the circuitry configured to generate data including at least one type of information on a time length of walking exercise, number of steps in a predetermined period, average walking speed of predetermined time points or of a predetermined period, or an intensity of the walking exercise, from the motion information.

19. The information processing apparatus according to claim 2, further comprising:
the circuitry configured to
generate evaluation information of a physical exercise from the motion information; and
generate the utterance information by using the evaluation information.

20. An information processing system, comprising:
a motion information obtaining device configured to obtain motion information related to a physical exercise of a user;
a reaction information obtaining device configured to obtain reaction information related to a reaction of the user; and
an information processing apparatus configured to output utterance information related to exercise support for the user based on the reaction information and the motion information.

21. An information processing system, comprising:
a wearable terminal to be worn by a user; and
a display terminal including a display on which a video related to a physical exercise is displayed, wherein
the wearable terminal includes a transmitter configured to transmit reaction information to the display terminal, and
the display terminal includes circuitry configured to
input motion information related to a physical exercise of the user;
input the reaction information received from the wearable terminal; and
output utterance information related to exercise support for the user based on the reaction information and the motion information.

22. The information processing system according to claim 21, wherein
the wearable terminal includes
a first sensor configured to obtain the reaction information related to a reaction of the user; and
a second sensor configured to obtain the motion information related to the physical exercise of the user, and
the transmitter is configured to transmit the reaction information obtained by the first sensor and the motion information obtained by the second sensor to the display terminal.

23. The information processing system according to claim 21, wherein
the display terminal includes an image capturer configured to capture an image, and
the motion information is movement information related to movement of the user obtained by analyzing the image.

24. A non-transitory computer-readable recording medium storing a program causing a computer to perform a method, the method comprising:
inputting motion information related to a physical exercise of a user,
inputting reaction information related to a reaction of the user, and
outputting utterance information related to exercise support for the user based on the reaction information and the motion information.

25. A method executed by computer processing, the method comprising:
inputting motion information related to a physical exercise of a user;
inputting reaction information related to a reaction of the user; and
outputting utterance information related to exercise support for the user based on the reaction information and the motion information.
